(19) United States
(12) Patent Application Publication
(10) Pub. No.: US 2025/0186149 A1
(43) Pub. Date: Jun. 12, 2025

(54) END EFFECTOR UNIT, SURGICAL TOOL DEVICE, ARM DEVICE, AND MASTER-SLAVE SYSTEM

(71) Applicant: SONY GROUP CORPORATION, TOKYO (JP)

(72) Inventor: KAZUHITO WAKANA, TOKYO (JP)

(21) Appl. No.: 18/843,512

(22) PCT Filed: Jan. 20, 2023
(86) PCT No.: PCT/JP2023/001742

§ 371 Date: Sep. 3, 2024

(30) Foreign Application Priority Data
Mar. 14, 2022 (JP) 2022-039748

(51) Int. Cl. A61B 34/37; A61B 34/00; B25J 3/00; B25J 9/10

(52) U.S. Cl. CPC A61B 34/37; B25J 3/00; B25J 9/104; A61B 2034/715

(57) ABSTRACT

Provided is an end effector unit that is exchangeably attached to a drive unit and operates by a driving force transmitted from the drive unit. The end effector unit includes a cable having one end on a tip end side connected to an end effector, a pulley configured to fold back the cable extending toward a root side in a tip end direction, and a linear motion transmission device having one degree of freedom in linear traveling in a longitudinal direction and connected to the other end of the cable folded back by the pulley. The linear motion transmission device and the pulley are alternately arranged in a circumferential direction around a longitudinal axis, and are reduced in size and diameter.

### TECHNICAL FIELD

The technology disclosed in the present specification (hereinafter, “the present disclosure”) relates to an end effector unit that is exchangeably attached to a drive unit and operates an end effector at a distal end by a driving force transmitted from the drive unit, a surgical tool device including a surgical tool unit having the end effector as a surgical tool for surgery and the drive unit, an arm device that supports the surgical tool device, and a master-slave system that annularly operates the arm device.

### BACKGROUND ART

For example, a surgical robot used in the medical field includes an arm device on which an end effector including a surgical tool and an observation device (an endoscope or the like) is mounted at a distal end. For example, there has been proposed a robotic surgical assembly including a slave manipulator including an actuator and a transmission mechanism, and a surgical instrument including a joint subassembly such as a medical instrument at a distal end, the surgical instrument being detachably attached to the slave manipulator (see Patent Document 1).

### CITATION LIST

### Patent Document

Patent Document 1: Japanese Translation of PCT International Application Publication No. 2020-516430
Patent Document 2: Japanese Patent Application Laid-Open No. 2021-41037
Patent Document 3: Japanese Patent Application Laid-Open No. 2021-41038

### SUMMARY OF THE INVENTION

### Problems to be Solved by the Invention

An object of the present disclosure is to provide an end effector unit that is exchangeably attached to a drive unit and operates an end effector at a distal end by a driving force transmitted from the drive unit, a surgical tool device including a surgical tool unit having the end effector as a surgical tool for surgery and the drive unit, an arm device that supports the surgical tool device, and a master-slave system that annularly operates the arm device.

### Solutions to Problems

The present disclosure has been made in view of the above-described problem, and the first aspect thereof is an end effector unit including:
a cable having one end on a tip end side connected to an end effector;
a pulley configured to fold back the cable extending toward a root side in a tip end direction; and
a linear motion transmission device having one degree of freedom in linear traveling in a longitudinal direction and connected to the other end of the cable folded back by the pulley.

The end effector unit according to the first aspect is reduced in size and diameter by devising a layout of the cable and the pulley to bring the linear motion transmission device including a rod or the like closer to a center of a circle.

Furthermore, the linear motion transmission device includes a rod having one degree of freedom in linear traveling in the longitudinal direction. The linear motion transmission device further includes a rotation suppression device that suppresses rotation of the rod about a longitudinal axis.

Furthermore, the second aspect of the present disclosure is a surgical tool device including:
a surgical tool unit including a surgical tool supported at a distal end, a cable having one end on a tip end side connected to the surgical tool, a pulley that folds back the cable extending to a root side in a tip end direction, and a linear motion transmission device having one degree of freedom in linear traveling in a longitudinal direction and connected to the other end of the cable folded back by the pulley; and
a drive unit to which the surgical tool unit is attached and configured to drive the linear motion transmission device.

Furthermore, the third aspect of the present disclosure is an arm device including:
a surgical tool device including a surgical tool unit including a surgical tool supported at a distal end, a cable having one end on a tip end side connected to the surgical tool, a pulley that folds back the cable extending to a root side in a tip end direction, and a linear motion transmission device having one degree of freedom in linear traveling in a longitudinal direction and connected to the other end of the cable folded back by the pulley, and a drive unit to which the surgical tool unit is attached and configured to drive the linear motion transmission device; and
an arm of an articulated link structure that supports the surgical tool device.

Furthermore, the fourth aspect of the present disclosure is a master-slave system including:
a slave device including a surgical tool device including a surgical tool unit including a surgical tool supported at a distal end, a cable having one end on a tip end side connected to the surgical tool, a pulley that folds back the cable extending to a root side in a tip end direction, and a linear motion transmission device having one degree of freedom in linear traveling in a longitudinal direction and connected to the other end of the cable folded back by the pulley, and a drive unit to which the surgical tool unit is attached and configured to drive the linear motion transmission device, and an arm of an articulated link structure that supports the surgical tool device; and
a master device configured to operate the surgical tool device and the arm.

However, the term “system” referred to here indicates a logical assembly of multiple devices (or functional modules that implement specific functions), and it does not matter whether or not each of the devices or functional modules is in a single housing. That is, one device including multiple components or functional modules and an assembly of multiple devices correspond to the “system”.

### Effects of the Invention

According to the present disclosure, it is possible to provide an end effector unit that is exchangeably attached to a drive unit and operates an end effector at a distal end by a driving force transmitted from the drive unit by a linear motion transmission device, a surgical tool device including a surgical tool unit having the end effector as a surgical tool for surgery and the drive unit, an arm device that supports the surgical tool device, and a master-slave system that annularly operates the arm device.

Note that the effects described in the present specification are merely examples, and the effects to be brought by the present disclosure are not limited thereto. Furthermore, the present disclosure further provides additional effects in addition to the effects described above in some cases.

Still other objects, features, and advantages of the present disclosure will become apparent from a more detailed description based on embodiments as described later and the accompanying drawings.

### BRIEF DESCRIPTION OF DRAWINGS

FIG. 1 is a view illustrating a surgical tool device 100 in a state where a surgical tool unit 101 is attached to a drive unit 103.

FIG. 2 is a view illustrating the surgical tool device 100 in a state where the surgical tool unit 101 is separated from the drive unit 103.

FIG. 3 is a view illustrating a state in which the drive unit 103 in a state in which the surgical tool unit 101 is attached is further mounted on an arm device 300.

FIG. 4 is a view illustrating an internal configuration by a cross-sectional view of the surgical tool unit 101.

FIG. 5 is a view illustrating an internal configuration by a cross-sectional view of an adapter unit 102 and the drive unit 103 cut along a plane including a longitudinal direction.

FIG. 6 is a view illustrating an image in which the surgical tool unit 101 is attached to the drive unit.

FIG. 7 is a view illustrating a specific configuration of the surgical tool unit 101.

FIG. 8 is a view illustrating a cross-sectional configuration of the surgical tool unit 101 illustrated in FIG. 7.

FIG. 9 is a perspective view of an inner base 703.

FIG. 10 is a view illustrating a cross section of the surgical tool unit 101 cut along a plane 750 orthogonal to the longitudinal direction.

FIG. 11 is a perspective view of a root side of the surgical tool unit 101 cut along the plane 750.

FIG. 12 is an exploded view of the surgical tool unit 101 exploded in the longitudinal direction.

FIG. 13 is a perspective view of a rod 713.

FIG. 14 is a perspective view of a state in which a vicinity of a pulley support portion 940 of the inner base 703 is viewed from a root side.

FIG. 15 is a back view of the surgical tool unit 101 (or a joint unit 704) illustrated in FIG. 7 as viewed from the root side.

FIG. 16 is cross-sectional views of the surgical tool device 100.

FIG. 17A is a cross-sectional view of the adapter unit 102.

FIG. 17B is a perspective view of the adapter unit 102.

FIG. 18 is an exploded view of the adapter unit 102 exploded in the longitudinal direction.

FIG. 19 is an exploded view of the adapter unit 102 exploded in the longitudinal direction.

FIG. 20 is an enlarged view of a vicinity of a root of the surgical tool unit 101 attached to the adapter unit 102.

FIG. 21 is front views illustrating respective states of the surgical tool unit 101 before attachment, in a middle of attachment, and at an attachment position to the adapter unit 102.

FIG. 22 is views illustrating a procedure of attaching the surgical tool unit 101 to the adapter unit 102 (before attachment).

FIG. 23 is views illustrating a procedure of attaching the surgical tool unit 101 to the adapter unit 102 (in the middle of attachment).

FIG. 24 is views illustrating a procedure of attaching the surgical tool unit 101 to the adapter unit 102 (in the middle of attachment).

FIG. 25 is views illustrating a procedure of attaching the surgical tool unit 101 to the adapter unit 102 (in the middle of attachment).

FIG. 26 is views illustrating a procedure of attaching the surgical tool unit 101 to the adapter unit 102 (at the time of completion of attachment).

FIG. 27 is views illustrating a procedure of detaching the surgical tool unit 101 from the adapter unit 102 (before detachment).

FIG. 28 is views illustrating a procedure of detaching the surgical tool unit 101 from the adapter unit 102 (in the middle of detachment).

FIG. 29 is views illustrating a procedure of detaching the surgical tool unit 101 from the adapter unit 102 (in the middle of detachment).

FIG. 30 is views illustrating a procedure of detaching the surgical tool unit 101 from the adapter unit 102 (in the middle of detachment).

FIG. 31 is views illustrating a procedure of detaching the surgical tool unit 101 from the adapter unit 102 (at the time of completion of detachment).

FIG. 32 is a view illustrating the degree of freedom configuration example of the arm device 300.

FIG. 33 is views illustrating a series of operations in which the arm device 300 pans the surgical tool device 100.

FIG. 34 is views illustrating a series of operations in which the arm device 300 tilts the surgical tool device 100 with respect to a main body of the arm device 300.

FIG. 35 is views illustrating a series of operations in which the arm device 300 tilts the surgical tool device 100 at a current position.

FIG. 36 is a diagram illustrating a functional configuration example of a master-slave system 3600.

### MODE FOR CARRYING OUT THE INVENTION

Hereinafter, the present disclosure will be described in the following order with reference to the drawings.
A. Outline
B. Overall Configuration
C. Basic Configuration and Operation of Surgical Tool Device
D. Detailed Configuration of Surgical Tool Unit
E. Diameter Reduction Structure of Surgical Tool Unit
F. Attaching/Detaching Structure, Attaching Procedure, and Detaching Procedure of Surgical Tool Unit
   F-1. Attaching/Detaching Structure of Surgical Tool Unit
   F-2. Attaching Procedure of Surgical Tool Unit
   F-3. Detaching Procedure of Surgical Tool Unit
G. Arm Device
H. Master-slave System

### A. Outline

The present disclosure relates to an end effector unit that is interchangeably attached to a drive unit and operates an end effector at a distal end by a driving force transmitted from the drive unit by a linear motion transmission device. The drive unit to which the end effector unit is attached is mounted on, for example, an arm device (also referred to as a robot or a manipulator) and used to perform work. In the present specification, embodiments in which the present disclosure is applied to the medical field will be especially mainly described. In such embodiments, the end effector is a surgical tool. Hereinafter, the end effector unit is also referred to as a “surgical tool unit”, and is also referred to as a “surgical tool device” in a state where the surgical tool unit is attached to the drive unit.

The surgical tool device is mounted on, for example, an arm device (also referred to as a surgical robot or a surgical manipulator) and used for surgery. Furthermore, the surgical tools are various types of medical instruments such as a pair of forceps, a pneumoperitoneum tube, an energy treatment tool, a pair of tweezers, and a retractor, for example. Therefore, an operation of preparing a plurality of types of surgical tool units including medical instruments of different types at the distal end, replacing the surgical tool unit attached to the drive unit as necessary during the surgery, and further, automatically replacing the surgical tool unit by the robot is conceivable.

In order for the robot itself to automatically replace the surgical tool, it is desirable that the plurality of surgical tool units to be replaced is installed on a mounting table such as a “surgical tool stand”. When the surgical tool units are reduced in diameter and size, it is easy to arrange the surgical tool units to be replaced and the surgical tool stand near the robot.

Incidentally, in the case of the robotic surgical assembly disclosed in Patent Document 1, a coupling device 93 that restrains a transmission device 54 so as to have one degree of freedom in linear motion with respect to a frame 52 is configured by a linear bearing, and the size thereof is larger than that of a simple bearing. Therefore, it seems difficult to reduce the diameter of the joint subassembly.

Therefore, in the present disclosure, the diameter of the surgical tool unit is reduced by using a linear motion transmission structure for the transmission device that transmits the driving force of the drive unit to the surgical tool at the distal end and enhancing slidability of the linear motion transmission structure. By reducing the diameter of the surgical tool unit, an arm (and the drive unit) at a connection destination and the surgical tool stand can be reduced in size and easily arranged near the robot. Furthermore, when the robot automatically replaces the surgical tool unit, the robot can perform accurate attachment even if the robot roughly inserts the root of the surgical tool unit into the tip end of the drive unit.

### B. Overall Configuration

In this item B, an overall configuration of the surgical tool device including the surgical tool unit and the drive unit will be described.

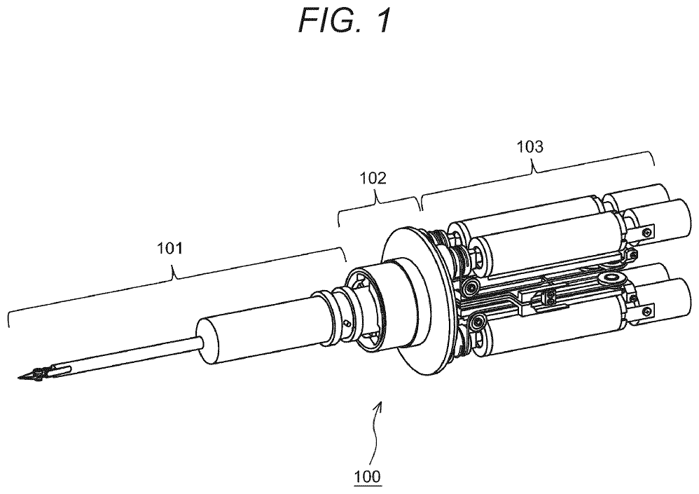

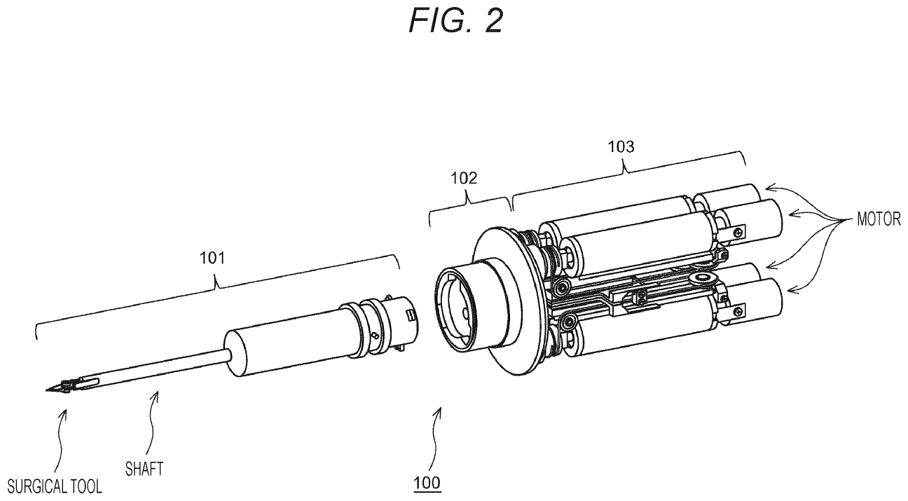

FIG. 1 illustrates a surgical tool device 100 in a state where a surgical tool unit 101 is attached to a drive unit 103. Furthermore, FIG. 2 illustrates the surgical tool device 100 in a state where the surgical tool unit 101 is separated from the drive unit 103. The surgical tool device 100 includes the surgical tool unit 101, an adapter unit 102, and the drive unit 103 in this order from a distal end side. Note that, in actual surgery, it is necessary to separate the surgical tool unit 101 arranged in a clean region from an unclean region on a root side, and it is assumed that a drape (not illustrated) is arranged and used between the surgical tool unit 101 and the adapter unit 102 or between the adapter unit 102 and the drive unit 103.

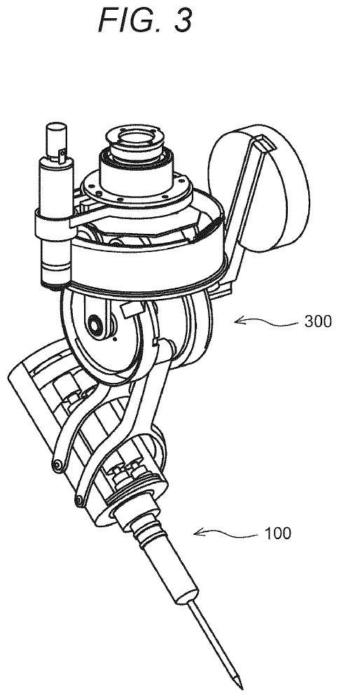

As illustrated in FIGS. 1 and 2, the adapter unit 102 for receiving (or inserting) the root side of the surgical tool unit 101 is attached to a tip end of the drive unit 103. Therefore, it can be said that the surgical tool unit 101 is attached to the drive unit 103 via the adapter unit 102. Furthermore, FIG. 3 illustrates a state in which the surgical tool device 100 in the state where the surgical tool unit 101 is attached to the drive unit 103 is further mounted on an arm device 300. The arm device 300 can cause the surgical tool unit 101 attached to the drive unit 103 to perform a pan operation and a tilt operation, which will be described below.

The surgical tool unit 101 includes a surgical tool and a hollow shaft that supports the surgical tool at the tip end (or the distal end). The surgical tools include various types of medical instruments such as a pair of forceps, a pneumoperitoneum tube, an energy treatment tool, a pair of tweezers, and a retractor, for example. However, hereinafter, for convenience, an embodiment specialized for a pair of forceps including a pair of jaws that opens and closes the surgical tool will be mainly described.

In the present embodiment, a structure of the forceps applied as an end effector, that is, as a surgical tool will be briefly described. The forceps includes a pair of jaws. Each of the jaws is rotatably supported about an opening/closing axis (or a yaw axis) near the tip end of the shaft, and a torsion spring that applies in advance an opening force acting in a direction of opening (or in a direction where the jaws are separated from each other) about an opening/closing axis between the jaws is incorporated in the opening/closing axis (or the yaw axis). Then, the forceps including the pair of jaws is supported by the distal end of the shaft so as to be rotatable about a pitch axis (wrist) orthogonal to each of the opening/closing axis and a longitudinal axis of the shaft.

A cable (not illustrated in FIGS. 1 and 2) for transmitting a driving force generated by the drive unit 103 to the surgical tool at the distal end is inserted through the shaft of the surgical tool unit 101. In the case where the surgical tool includes a pair of jaws as described in the preceding paragraph, a total of four cables including one cable for pulling each of the jaws in a closing (or approaching the other jaw) direction about the opening/closing axis and one cable for rotating the entire forceps including the pair of jaws for each of forward and backward rotations about the pitch axis (wrist) are inserted into the shaft.

Furthermore, as will be described below, each cable inserted into the shaft is wound around a pulley on the root side of the surgical tool unit 101 (or the shaft) and folded back in the direction of the distal end (or the tip end), and then connected to a rod as a linear motion transmission device (or the vicinity of the end is fixed). A corresponding rod is required for each cable, and a total of four rods are arranged on the root side of the surgical tool unit 101. Each rod has only one degree of freedom to linearly move in the longitudinal direction of the surgical tool unit 101 (or the shaft). When the rod advances to a proximal side, the cable connected to the rod is pulled toward the root side to transmit the driving force.

As can be seen from FIGS. 1 and 2, the root side of the surgical tool unit 101 is attached to the tip end side of the drive unit 103 via the adapter unit 102. As long as each rod on the surgical tool unit 101 side can be attached so as to just come into contact with the corresponding rod on the drive unit 103 side, the method and structure of attaching the surgical tool unit 101 to the adapter unit 102 are not particularly limited.

The drive unit 103 includes the number of sets of a motor that generates the driving force and a rotation-to-linear motion conversion mechanism that converts a rotational motion of the motor into a linear motion, the number corresponding to the number of cables on the surgical tool unit 101 side. Furthermore, each motor includes an encoder and a brake. As described above, since the surgical tool unit 101 includes four cables, the drive unit 103 is equipped with four sets of the motors and the rotation-to-linear motion conversion mechanisms. Four cylindrical parts arranged on the proximal side of the drive unit 103 illustrated in FIGS. 1 and 2 are the respective motors. The rotation-to-linear motion conversion mechanism arranged for each motor is not illustrated in FIGS. 1 and 2.

### C. Basic Configuration and Operation of Surgical Tool Device

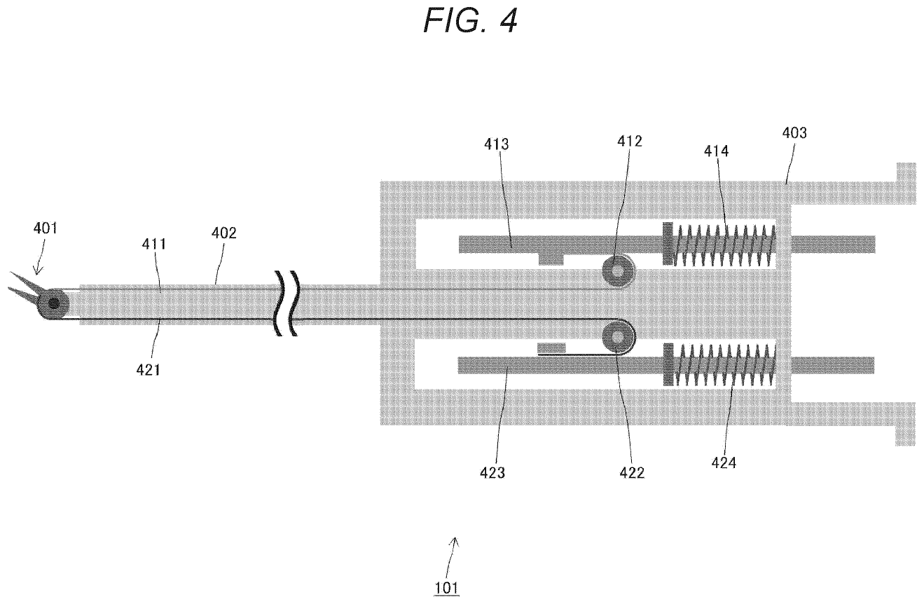

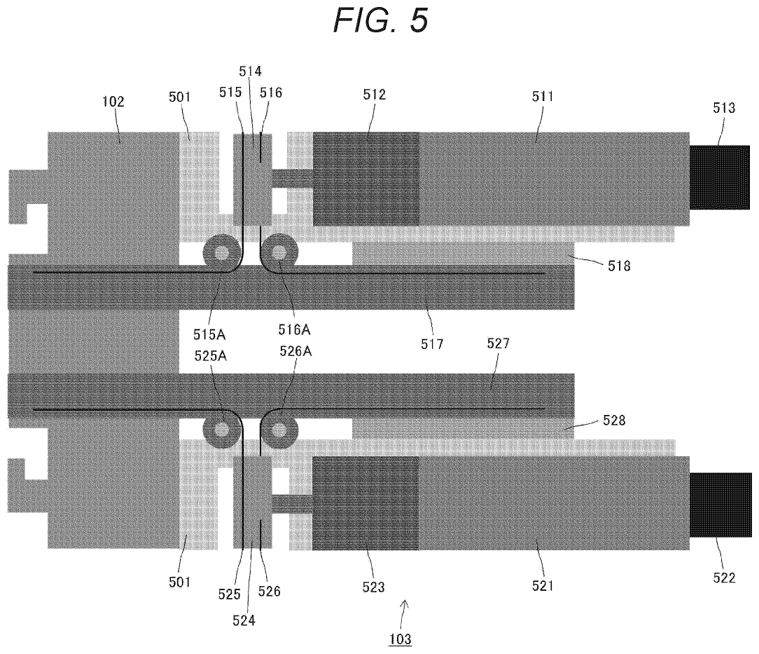

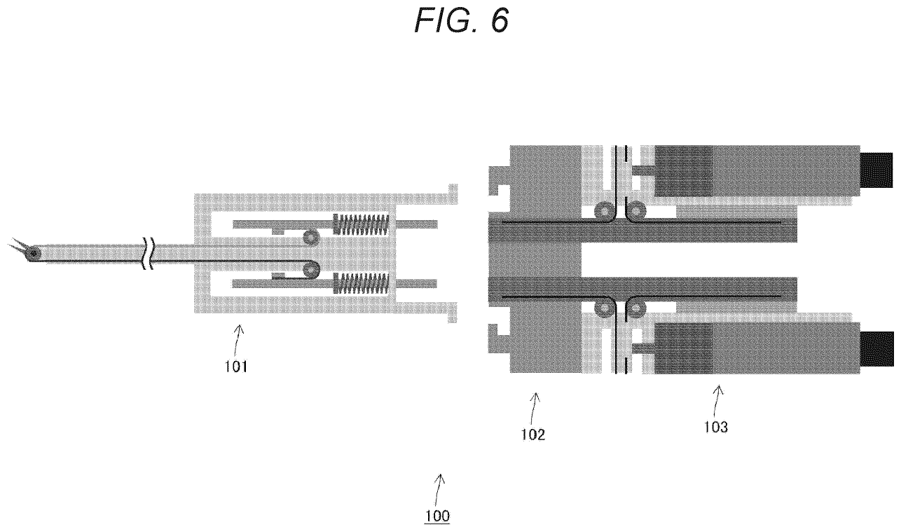

In this item C, a basic configuration and operation of the surgical tool device 100 will be described. As described in the above item B, the surgical tool device 100 includes the surgical tool unit 101 and the drive unit 103, and the surgical tool unit 101 is exchangeably attached to the drive unit 103 via the adapter unit 102. FIG. 4 illustrates an internal configuration by a cross-sectional view of the surgical tool unit 101 cut along a plane including the longitudinal direction, and FIG. 5 illustrates an internal configuration by a cross-sectional view of the adapter unit 102 and the drive unit 103 cut along a plane including the longitudinal direction. Note that, for convenience, an xy axis is defined as illustrated in FIG. 4. The x-axis corresponds to the longitudinal axis. Furthermore, FIG. 6 illustrates an image in which the surgical tool unit 101 illustrated in FIG. 4, and the adapter unit 102 and the drive unit 103 illustrated in FIG. 5 are arranged in the longitudinal direction, and the surgical tool unit 101 is attached to the drive unit 103.

First, an operation principle of the linear motion transmission mechanism mounted on the surgical tool unit 101 will be described with reference to FIG. 4. The surgical tool unit 101 includes a surgical tool 401, a hollow shaft 402 that supports the surgical tool 401 at the tip end (or the distal end), and a surgical tool unit base 403 that supports the shaft 402 and is coupled to the adapter 102 on the root side. FIG. 4 illustrates the internal configuration of each of the shaft 402 and the surgical tool unit base 403 by the cross-sectional view of the surgical tool unit 101 cut along a plane including the longitudinal direction.

As described above, the surgical tool unit 101 includes the surgical tool 401 including a pair of jaws, and the total of four cables including two cables 411 and 421 that pull the respective jaws about the opening/closing axis, and the two cables (not illustrated in FIG. 4) that rotate the surgical tool 401 (or the opening/closing axis of the jaws) forward and backward about the pitch axis (wrist). The surgical tool 401 has, for example, three degrees of freedom in opening and closing (or grip) of each jaw, and rotations about the opening/closing axis (or the yaw axis) and about the pitch axis of the forceps, but details are omitted here for simplification of description. Note that the number of cables necessary for driving the surgical tool 401 differs depending on the degree of freedom configuration of the surgical tool 401 and the like. FIG. 4 illustrates the two cables 411 and 421 and two linear motion transmission devices corresponding to the respective cables 411 and 421 for simplification of the drawing. Furthermore, hereinafter, for convenience, the description will be given, not particularly limiting whether each of the cables 411 and 421 relates to any degree of freedom in the opening and closing (or grip) of the surgical tool 401 (jaws) as the end effector, and the rotations of the surgical tool 401 about the yaw axis and the pitch axis.

One end of the tip end side (or distal side) of the cable 411 is connected to a capstan (not illustrated) related to any degree of freedom in the opening and closing (or grip), pitch, and yaw of the surgical tool 401 (jaw) as an end effector. Furthermore, the other end extending to the root side (or proximal side) of the cable 411 is inserted into the shaft 402, then drawn into the surgical tool unit base 403, wound around a pulley 412 on the root side of the surgical tool unit 101 (or the shaft 402), folded back in the direction of the distal end (or tip end), and then connected to a rod 413 via a cable coupling unit (described below) (or the vicinity of the end is fixed). The rod 413 is supported by the surgical tool unit base 403 so as to slide only in one degree of freedom in linear traveling in the x-axis direction, that is, the longitudinal direction. Furthermore, the pulley 412 is an idler pulley, and is rotatably supported by the surgical tool unit base 403.

As will be described below with reference to FIG. 5, the rod 413 retreats and advances in the x-axis direction by the driving force transmitted from the drive unit 103 side. Then, when the rod 413 retreats in the x-axis direction (in other words, advances in a distal direction), the cable 411 connected to the rod 413 is pulled and can transmit the driving force to the capstan related to any degree of freedom of the surgical tool 401 as an end effector. Therefore, the rod 413 serves as a linear motion transmission device.

This is because when the rod 413 rotates about its own longitudinal axis, the cable 411 is wound around the rod 413, and cannot accurately drive the surgical tool 401 according to a linear traveling amount of the rod 413 in the longitudinal direction. Therefore, the rod 413 is equipped with a rotation suppression device so as to suppress the rotation about the longitudinal axis and to operate only in one degree of freedom in linear traveling in the longitudinal direction, but this point will be described below in detail.

Furthermore, a spring 414 that applies a force to push the rod 413 in the tip end direction is arranged on the root side of the rod 413 so that the cable 411 is not loosened even in a state where the surgical tool unit 101 is separated from and independent of the drive unit 103. The rod 413 passes through the spring 414, and one end of the spring 414 is fixed to the surgical tool unit 403 and the other end is fixed to the rod 413. Therefore, when the rod 413 is pushed in the tip end direction by an elastic force of the spring 414, a pre-tension force is applied to the cable 411 folded back by the pulley 412 and coupled to the rod 413, so that the cable is not loosened.

The cable 421 is similarly to the cable 411, one end on the tip end side (or distal side) of the cable 421 is connected to a capstan (not illustrated) related to any degree of freedom of the surgical tool 401 (jaw) as an end effector, and the other end extending to the root side (or proximal side) of the cable 421 is inserted into the shaft 402, then drawn into the surgical tool unit base 403, wound around a pulley 422 on the root side of the surgical tool unit 101 (or the shaft 402) and folded back, and then connected to a rod 423 via a cable coupling unit (described below) (or the vicinity of the end is fixed). The rod 413 is supported by the surgical tool unit base 403 so as to slide only in one degree of freedom in linear traveling in the x-axis direction, that is, the longitudinal direction. Furthermore, the pulley 422 is rotatably supported by the surgical tool unit base 403. The rod 423 as a linear motion transmission device retreats and advances in the x-axis direction by the driving force transmitted from the drive unit 103 side. When the rod 423 retreats in the x-axis direction (in other words, advances in the distal direction), the cable 421 connected to the rod 423 is pulled and can transmit the driving force to the surgical tool 401. The rod 423 is equipped with a rotation suppression device so as to suppress the rotation about the longitudinal axis and to operate only in one degree of freedom in linear traveling in the longitudinal direction (to be described below). Furthermore, a spring 424 that applies a force to push the rod 423 in the tip end direction is arranged on the root side of the rod 423 so that the cable 421 is not loosened even in a state where the surgical tool unit 101 is separated from and independent of the drive unit 103. The rod 423 passes through the spring 424, and one end of the spring 424 is fixed to the surgical tool unit 403 and the other end is fixed to the rod 423.

Note that FIG. 4 illustrates the pulleys 412 and 422 arranged at positions where the pulleys do not interfere with other components and are easy to see, for convenience of describing the basic configuration of the surgical tool unit 101. However, the actual arrangement positions are different from those in FIG. 4. In actual implementation, the arrangement positions of the pulleys 412 and 422 are determined so that the surgical tool unit 101 (more specifically, the surgical tool unit base 403) can be reduced in size and diameter, but details will be described below.

Furthermore, it should be understood that a linear motion transmission device similar to the cables 411 and 421 illustrated in FIG. 4 is provided in the cable (not illustrated) used according to the degree of freedom of the surgical tool 401.

Next, an operation principle of the rotation-to-linear motion conversion mechanism mounted on the drive unit 103 will be described with reference to FIG. 5. The drive unit 103 includes the number of sets of the motor that generates the driving force and the rotation-to-linear motion conversion mechanism that converts a rotational motion of the motor into a linear motion, the number corresponding to the number of cables on the surgical tool unit 101 side. The number of cables on the surgical tool unit 101 side differs depending on the degrees of freedom configuration of the surgical tool 401 and the like, and accordingly, the number of motors and rotation-to-linear motion conversion mechanisms provided on the drive unit 103 side also differs. FIG. 5 illustrates the configuration of the motors and the rotation-to-linear motion conversion mechanism for driving the cables 411 and 421 of the surgical tool unit 101 illustrated in FIG. 4 by a cross-sectional view of the drive unit 103 cut along a plane including the longitudinal direction. Furthermore, in FIG. 5, to simplify the drawing, the adapter unit 102 is drawn integrally with a drive unit base 501 on the drive unit 103 side. Note that, for convenience, the same xy axes as those in FIG. 4 are also defined in FIG. 5.

A motor 511 is fixed in the drive unit 103 via the drive unit base 501 on the proximal side of the drive unit 103 for driving the rod 413 that pulls the cable 411 on the surgical tool unit 101 side. In the motor 511, a speed reducer 512 is attached to an output end, and an encoder 513 that measures a rotation angle of a rotation shaft (not illustrated) of the motor 511 is attached to an end surface at an opposite side of the output end. In consideration of reduction in size, an incremental type is adopted as the encoder 513, but an absolute type encoder may be adopted as a matter of course. Furthermore, the motor 511 may further include a brake (not illustrated).

The rotation-to-linear motion conversion mechanism that converts the rotation of the motor 511 into the linear motion includes a motor capstan 514 attached to an output shaft of the motor 511 (or the speed reducer 512), a pair of cables 515 and 516 having one ends wound around the motor capstan 514 in opposite directions to each other, a rod 517, and a linear guide 518 that guides the rod 517 to slide with respect to the drive unit base 501 only in one degree of freedom in linear motion in the x-axis direction, that is, the longitudinal direction with respect to the drive unit base 501. The other end of the cable 515 is rerouted from a circumferential direction of the motor capstan 514 to a negative direction of the x-axis (in the distal direction of the rod 517) via an idler pulley 515A and then connected to the distal side of the rod 517. Furthermore, the other end of the cable 516 is rerouted from the circumferential direction of the motor capstan 514 to a positive direction of the x-axis (in the proximal direction of the rod 517) via an idler pulley 516A and then connected to the proximal side of the rod 517. The linear guide 518 is fixed to the drive unit base 501 such that the direction of guiding the rod 517 coincides with the x-axis direction, that is, the longitudinal direction. Furthermore, each of the idler pulleys 515A and 516A is rotatably supported by the drive unit base 501.

When the motor 511 rotates forward, one cable 515 is wound around the motor capstan 514. As a result, the rotation is converted into a linear motion in which the rod 517 advances in the negative direction of the x-axis (or the distal side). Furthermore, when the motor 511 rotates backward, the other cable 516 is wound around the motor capstan 514. As a result, the rotation is converted into a linear motion in which the rod 517 retreats in the positive direction of the x-axis (or the proximal side).

When the surgical tool unit 101 is attached to the drive unit 103 via the adapter unit 102, the tip end of the rod 517 on the drive unit 103 side just comes into contact with the end of the rod 413 on the surgical tool unit 101 side. Therefore, when the rod 517 retreats and advances in the x-axis direction by the forward rotation and the backward rotation of the motor 511, the rod 413 also retreats and advances in the x-axis direction following the retreating and advancing. Then, when the rod 517 and the rod 413 retreat in the x-axis direction (in other words, advances in the distal direction), the cable 411 connected to the rod 413 is pulled and can linearly transmit the driving force to the capstan related to any degree of freedom of the surgical tool 401 as an end effector.

Furthermore, the motor 521 is arranged on the proximal side of the drive unit 103 for driving the cable 421 (or the rod 413). A speed reducer 522, an encoder 523, and a brake (not illustrated) are also attached to the motor 521. The rotation-to-linear motion conversion mechanism that converts the rotation of the motor 521 into the linear motion includes a motor capstan 524 attached to an output shaft of the motor 511 (or the speed reducer 522), a pair of cables 525 and 526 having one ends wound around the motor capstan 524 in opposite directions to each other, a rod 527, and a linear guide 528 that guides the rod 527 to slide only in one degree of freedom in linear traveling in the longitudinal direction with respect to the drive unit base 501. The other end of the cable 525 is rerouted from a circumferential direction of the motor capstan 524 to the negative direction of the x-axis (in the distal direction of the rod 527) via an idler pulley 525A and then connected to the distal side of the rod 517. Furthermore, the other end of the cable 526 is rerouted from the circumferential direction of the motor capstan 524 to a positive direction of the x-axis (in the proximal direction of the rod 527) via an idler pulley 526A and then connected to the proximal side of the rod 527. The linear guide 528 is fixed to the drive unit base 501 such that the direction of guiding the rod 527 coincides with the x-axis direction, that is, the longitudinal direction. Furthermore, each of the idler pulleys 525A and 526A is rotatably supported by the drive unit base 501.

When the motor 521 rotates forward, one cable 525 is wound around the motor capstan 524, and the rotation is converted into a linear motion in which the rod 527 advances in the negative direction of the x-axis (or the distal side). Furthermore, when the motor 521 rotates backward, the other cable 526 is wound around the motor capstan 524, and the rotation is converted into a linear motion in which the rod 527 retreats in the positive direction of the x-axis (or the proximal side).

When the surgical tool unit 101 is attached to the drive unit 103 via the adapter unit 102, the tip end of the rod 527 side just comes into contact with the end of the rod 423 on the surgical tool unit 101 side. Therefore, when the motor 521 rotates forward and the rod 527 retreats in the x-axis direction (in other words, advances in the distal direction), the cable 421 is pulled via the rod 423 and can linearly transmit the driving force to the capstan related to any degree of freedom of the surgical tool 401.

It should be understood that the motor (not illustrated) corresponding to each cable used according to the degree of freedom of the surgical tool 401 is also equipped with a rotation-to-linear motion transmission mechanism similar to the motors 511 and 521 described above, and linearly transmits the driving force to the surgical tool unit 101 side.

Note that, in the art, a ball screw is also known as the rotation-to-linear motion transmission mechanism that converts the rotational motion of the motor into the linear motion. In the case of using a ball screw, a problem of backlash and a problem of back drivability occur. When the back drivability is low, for example, it is difficult to detect an external force acting on the end effector such as the surgical tool on the root side. Furthermore, when using the ball screw, it is difficult to reduce the size and diameter of the surgical tool device.

In contrast, according to the rotation-to-linear motion conversion mechanism using cable driving of the rod as illustrated in FIG. 5, it is possible to realize backlashless and high back drivability, and for example, it is also suitable for a surgical robot that requires precise force control.

Furthermore, it is also possible to design such that the rotation-to-linear motion conversion mechanism is arranged on the surgical tool unit 101 side instead of the drive unit 103. However, as illustrated in FIGS. 4 and 5, the configuration in which the rotation-to-linear motion conversion mechanism is arranged in the drive unit 103 can realize the reduction in size and diameter of the surgical tool unit 101. When the surgical tool unit is small in size and diameter, for example, more surgical tool units can be installed in the surgical tool stand, and the robot can automatically replace the surgical tool unit in a short time.

Note that terms used in the present specification will be briefly described. The “capstan” and the “idler pulley” are both pulleys. A pulley used for cable layout adjustment and application of tension to the cable is referred to as an “idler pulley” in the present specification. Furthermore, a pulley used for application of power to the cable or conversely for conversion of a force from the cable into an axial force is referred to as a “capstan” in the present specification, and an input capstan and an output capstan are both pulleys used in this application.

### D. Detailed Configuration of Surgical Tool Unit

As described in the above item A, by reducing the size and diameter of the surgical tool unit, the arm (and the drive unit) at the connection destination and the surgical tool stand can be reduced in size, and more surgical tool units can be mounted on the surgical tool stand to improve the degree of integration. Furthermore, when the robot automatically replaces the surgical tool unit, the robot can perform accurate attachment even if the robot roughly inserts the root of the surgical tool unit into the tip end of the drive unit when the surgical tool unit is small in size and diameter. In this item D, a detailed configuration of the surgical tool unit for realizing the reduction in size and diameter will be described.

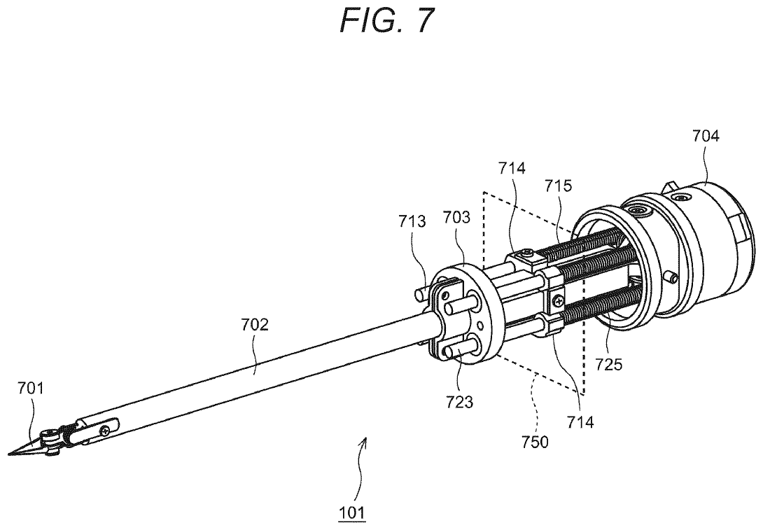

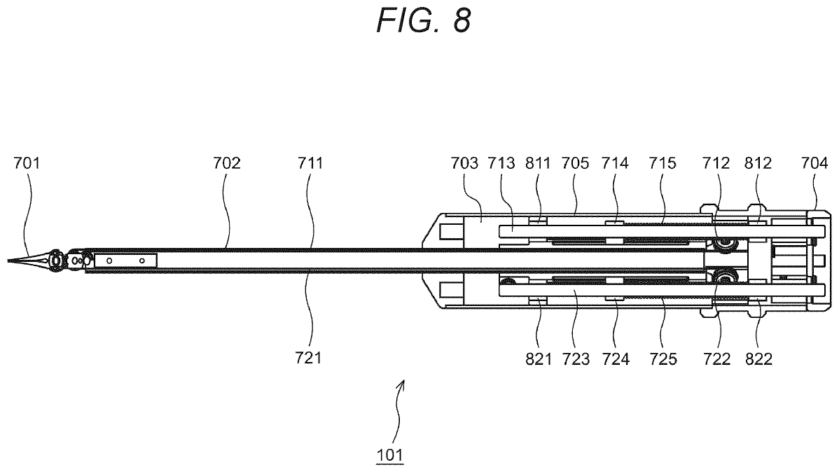

FIG. 7 is a perspective view illustrating a specific configuration of the surgical tool unit 101. Furthermore, FIG. 8 illustrates an internal configuration of the surgical tool unit 101 illustrated in FIG. 7 by a cross-sectional view taken along a plane including the longitudinal direction. The basic configuration and operation of the surgical tool unit 101 illustrated in FIGS. 7 and 8 are as described in the above item C with reference to FIG. 4.

The surgical tool unit 101 includes a surgical tool 701, a hollow shaft 702 that supports the surgical tool 701 at the tip end (or the distal end), and an inner base 703 that supports the shaft 702. The inner base 703 is fixed to a joint unit 704 for being joined to the adapter unit 102 on the root side (or the proximal side), and a periphery thereof is covered with a cylindrical case 705. Note that, in FIG. 7, illustration of the case 705 is omitted in order to clarify the internal structure.

The surgical tool 701 is not particularly limited, but may be, for example, a pair of forceps including a pair of jaws disclosed in Patent Document 2 or Patent Document 3. Each of the jaws is rotatably supported about the opening/closing axis (or the yaw axis) near the tip end of the shaft, and a torsion spring that applies in advance an opening force acting in the direction of opening (or in the direction where the jaws are separated from each other) about the opening/closing axis between the jaws is incorporated in the opening/closing axis (or the yaw axis). Then, the forceps including the pair of jaws is supported so as to be rotatable about the pitch axis (wrist) orthogonal to each of the opening/closing axis and the longitudinal axis of the shaft.

One end of the tip end side (or distal side) of the cable 711 is connected to a capstan (not illustrated in FIG. 7) related to any degree of freedom of the surgical tool 701 as an end effector. Furthermore, the other end extending to the root side (or the proximal side) of the cable 711 is inserted into the shaft 702, then drawn into the inner base 703, wound around a pulley 712 on the root side of the surgical tool unit 101 (or the shaft 702), folded back in the direction of the distal end (or the tip end), and then connected to a rod 713 via a cable coupling unit (described below) (or the vicinity of the end is fixed).

The cable 721 is similarly to the cable 711, one end on the tip end side (or the distal side) of the cable 721 is connected to a capstan (not illustrated) related to any degree of freedom of the surgical tool 701, and the other end extending to the root side (or the proximal side) of the cable 721 is inserted into the shaft 702, then drawn into the inner base 703, wound around a pulley 722 on the root side of the surgical tool unit 101 (or the shaft 702) and folded back, and then connected to a rod 723 via a cable coupling unit (described below) (or the vicinity of the end is fixed).

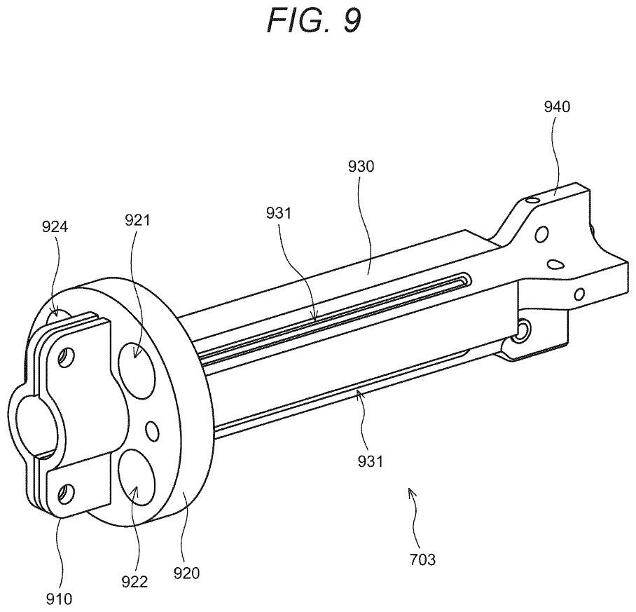

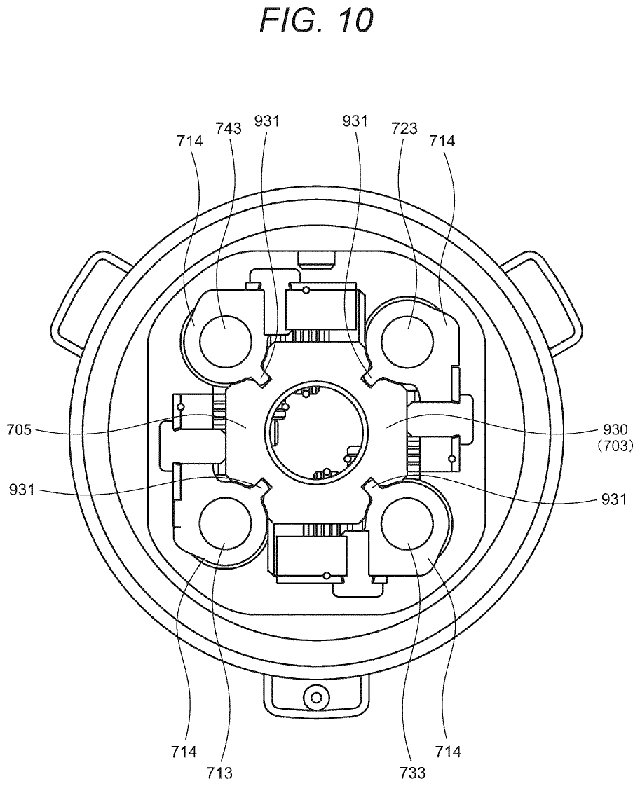

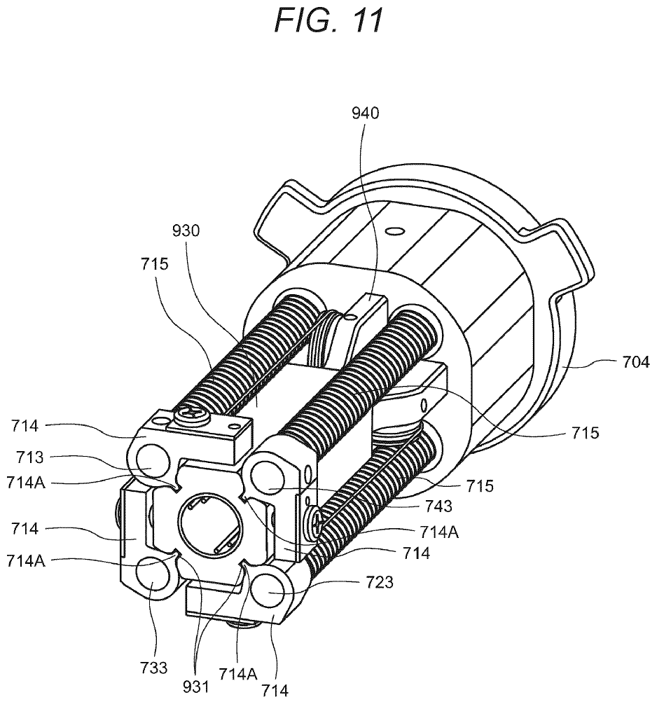

Further, FIG. 9 illustrates a perspective view of only the inner base 703 extracted from FIG. 7. Furthermore, FIG. 10 illustrates a cross-sectional view of the surgical tool unit 101 cut along a plane orthogonal to the longitudinal direction indicated by reference numeral 750 in FIG. 7. Furthermore, FIG. 11 illustrates a perspective view of the root side of the surgical tool unit 101 cut along a plane 750. The plane 750 cuts the surgical tool unit 101 just at the position where a rotation suppression device 714 (described below) is arranged. Furthermore, FIG. 12 is an exploded view of the surgical tool unit 101 exploded in the longitudinal direction.

As illustrated in FIG. 9, the inner base 703 includes, on the tip end side, a shaft connection portion 910 to which the shaft 702 is mounted, and a disk-shaped rod support portion 920 in which four rod insertion holes 921 to 924 through which four rods including the rods 713 and 723 are inserted in the longitudinal direction are drilled. The shaft connection portion 910 includes two symmetrical plate members each having a central attachment portion that forms a semi-cylindrical space, and by combining these plate members, a cylindrical space for attaching the shaft 702 is formed in the center. By inserting the root portion of the shaft 702 into the cylindrical space and then screwing the two plate members, the shaft connection portion 910 can sandwich the root portion of the shaft 702. Furthermore, in the inner base 703, a pulley support portion 940 that rotatably supports four idler pulleys for folding back the four cables including the pulleys 712 and 722 to the distal end side is arranged on the root side, and the rod support portion 920 and the pulley support portion 940 are connected via a frame 930 (the structure in which the pulley support portion 940 supports each idler pulley will be described below with reference to FIG. 14). Four guide grooves including a guide groove 931 are formed in the frame 930 along the longitudinal direction of the four rods including the rods 713 and 723. These guide grooves each has a length that covers a longitudinal movable range of the corresponding rod. Note that, for convenience of description, the inner base 703 is divided into the four portions 910 to 940, and each portion is named. However, it is assumed that the inner base 703 is manufactured as one integrated component. Of course, the inner base 703 may be configured by a combination of a plurality of components.

As can be seen from the cross-sectional view illustrated in FIG. 8, the rod 713 is supported at two positions of the rod insertion hole 921 of the rod support portion 920 of the inner base 703 and the joint unit 704 on the root side via bearings 811 and 812, respectively. Each of these bearings 811 and 812 is configured by a plain bearing and guides the linear motion of the rod 713 in the longitudinal direction. Note that the inner base 703 may be configured to be supported by plain bearings at three or more locations. Similarly, the rod 723 is supported at two positions of the rod insertion hole 921 of the rod support portion 920 of the inner base 703 and the joint unit 704 on the root side via bearings 821 and 822, respectively.

As described in the above item C with reference to FIG. 5, the rod 713 linearly moves (that is, retreats or advances) in the longitudinal direction by the driving force transmitted from the drive unit 103 side. Note that, since a specific configuration of the drive unit 103 is not directly related to the present disclosure, detailed description thereof is omitted in the present specification. When the rod 713 advances in the distal direction, the cable 711 connected to the rod 713 is pulled and can transmit the driving force to the capstan related to any degree of freedom of the surgical tool 701 as an end effector. Therefore, the rod 713 serves as a linear motion transmission device.

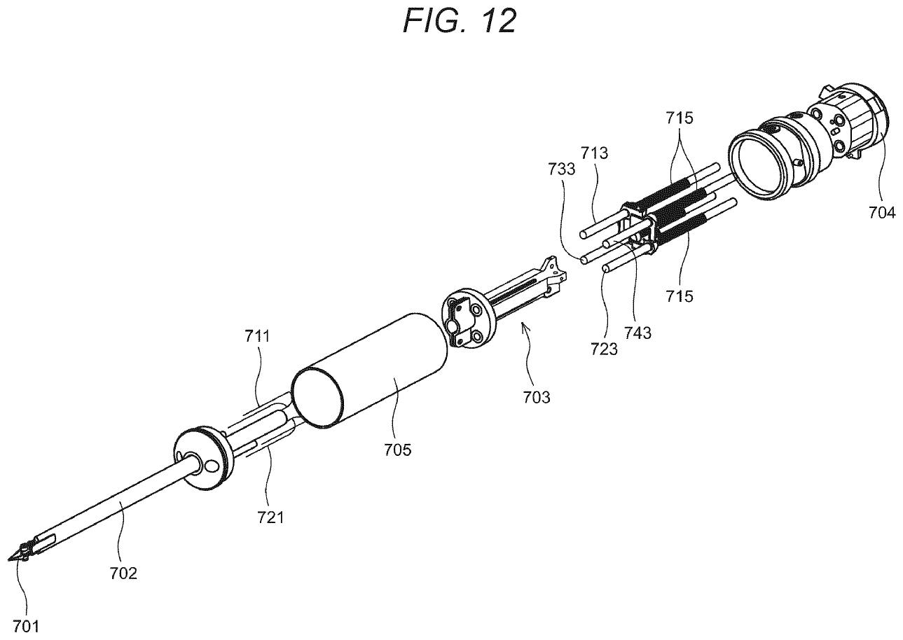

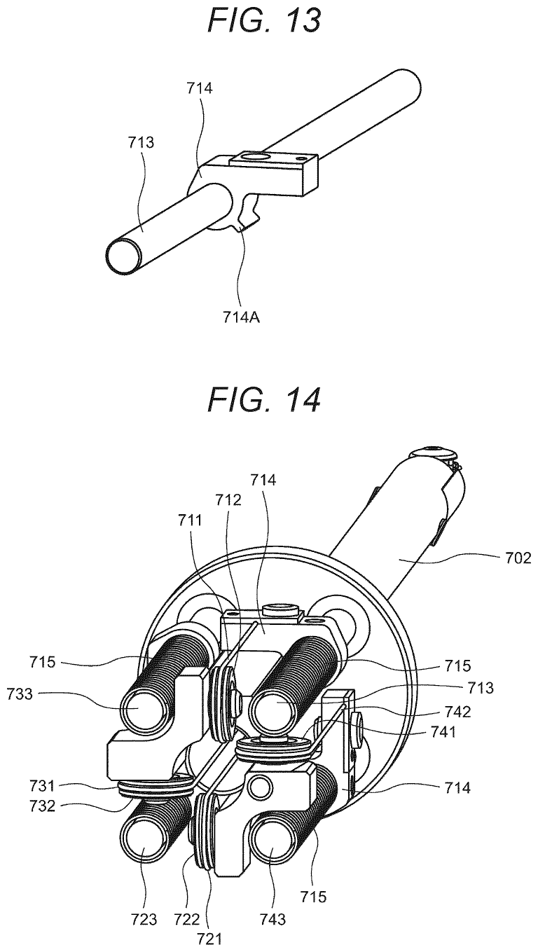

As can be seen from FIGS. 7 and 8, the rotation suppression device 714 is attached to the rod 713 at an intermediate position between two positions supported by the bearings 811 and 812. FIG. 13 illustrates a perspective view of only one rod 713 extracted from the surgical tool unit 101 illustrated in FIG. 7. A specific shape and structure of the rotation suppression device 714 will be more apparent with reference to FIG. 13. A main role of the rotation suppression device 714 is to suppress the rotation about the longitudinal axis when the rod 713 is guided by the bearings 811 and 812 and linearly moves. This is because if the rod 713 rotates about the longitudinal axis, the cable 711 having the other end fixed to the rod 713 is wound around the rod 713, and cannot accurately drive the surgical tool 701 according to the linear traveling amount of the rod 713 in the longitudinal direction.

As illustrated in FIG. 13, the rotation suppression device 714 includes a protrusion 714A protruding inward. Furthermore, as already described with reference to FIG. 9, the guide groove 931 is formed in the frame 930 along the longitudinal direction of the rod 713. In a state where the rod 713 to which the rotation suppression device 714 is attached is incorporated in the surgical tool unit 101, the protrusion 714A just enters the guide groove 931. The guide groove 931 has a length that covers the movable range of the rod 713 in the longitudinal direction. Therefore, the rod 713 linearly moves in the longitudinal direction while the protrusion 714A is guided by the guide groove 931, so that it is possible to suppress the rotation about the longitudinal axis of the rod 713.

Note that, by reversing a protrusion-recess relationship from the above description, when the frame 930 is provided with a line protrusion having the length covering the movable range of the rod 713 in the longitudinal direction, and a groove for sliding the line protrusion is formed on the rotation suppression device 714 side and guides the line protrusion in the groove on the rotation suppression device 714 side when the rod 713 linearly moves, the rotation suppression device 714 can similarly suppress the rotation of the rod 713 about the longitudinal axis.

Furthermore, the rotation suppression device 714 also serves as the cable coupling unit that connects (or fixes) the other end of the cable 711 wound around the pulley 712 and folded back in the direction of the distal end (or the tip end) (note that, in FIG. 13, illustration of the cable 711 is omitted in order to prevent complexity of the drawing). Note that the other end of the cable 711 may be fixed to the rod 713 by a cable coupling unit configured as a different component from the rotation suppression device 714. Furthermore, in the example illustrated in FIGS. 7 to 13, the rod 713 and the rotation suppression device 714 are configured as separate components, but the rod 713 and the rotation suppression device 714 may be configured as an integrated component.

Furthermore, a spring 715 that applies a force to push the rod 713 in the tip end direction is arranged on the root side of the rod 713 so that the cable 711 is not loosened even in a state where the surgical tool unit 101 is separated from and independent of the drive unit 703. Specifically, as can be seen from FIG. 12, the root side of the rod 713 is passed through the spring 715. In the assembled state of the surgical tool unit 101, the root side of the spring 715 comes into contact with a tip end surface of the joint unit 704, and the tip end side of the spring 715 comes into contact with the rotation suppression device 714. Therefore, the rotation suppression device 714 also serves as a contact surface of the spring 715. Then, in the state where the surgical tool unit 101 is separated from the drive unit 703, the rod 713 is pushed in the tip end direction by the elastic force of the spring 715, and a pre-tension force is applied to the cable 711 folded back by the pulley 712, so that the cable is not loosened.

Although the motion of the rod 713 and the rotation suppression device 714 related to the rod 713 have been mainly described above, it should be understood that each of the motion, the mechanism of the rotation suppression device, and the pre-tension applying spring of the cable is also similar for the rod 723, the cable 721, and the other three rods and cables.

### E. Diameter Reduction Structure of Surgical Tool Unit

In this item E, a structure for realizing the reduction in diameter of the surgical tool unit 101 illustrated in FIG. 7 will be described in detail.

As described in the above item D with reference to FIG. 9, the pulley support portion 940 arranged on the root side of the inner base 703 rotatably supports the four idler pulleys for folding back the four cables to the distal end side. FIG. 14 is a perspective view of a state in which a vicinity of the pulley support portion 940 of the inner base 703 is viewed from the root side. As can be seen from FIG. 14, the pulley support portion 940 has a cross-shaped cross section, and supports the idler pulley 712 for folding back the cable 711, the idler pulley 722 for folding back the cable 721, an idler pulley 732 for folding back a cable 731, and an idler pulley 742 for folding back a cable 741.

As can be seen from FIG. 14, on a plane orthogonal to the longitudinal direction, the rods 713, 723, 733, and 743 that respectively pull the plurality of (four in the present embodiment) cables 711, 721, 731, and 741, and the pulleys 712, 722, 732, and 742 that respectively fold back the cables 711, 721, 731, and 741 are arranged along a circumference centered on the longitudinal axis. By having the structure in which the cables passing through the shaft 702 are folded back in the distal direction by the pulleys, it is possible to lay out the cables and the pulleys together in a radial direction of the shaft 702 in a compact manner. Furthermore, it is possible to reduce the diameter of the surgical tool unit 101 (in particular, the portion of the case 705 including the rods) by devising the layout of the cables and the pulleys and bringing the rods for linearly moving the cables closer to the center of the circle.

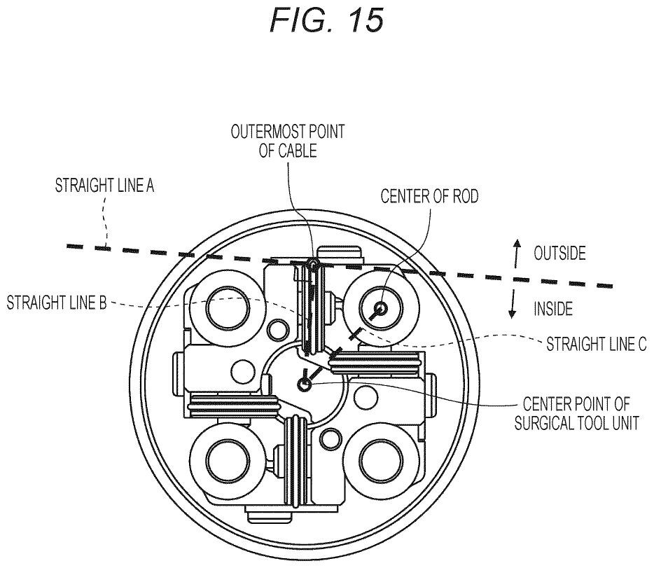

Here, the layout design of the cables and the pulleys for reducing the diameter will be specifically and geometrically described with reference to FIG. 15. FIG. 15 is a back view of the surgical tool unit 101 (or the joint unit 704) illustrated in FIG. 7 as viewed from the root side.

As a first condition for reducing the diameter of the surgical tool unit 101, a “straight line B” and a “straight line C” are defined, the straight line B connecting the longitudinal axis of the surgical tool unit 101 (hereinafter referred to as a “center point” of the surgical tool unit 101) and a point (hereinafter referred to as “outermost point”) farthest from the center point, of the cable folded back by the pulley, and the straight line C connecting the center point and a center of the rod, and the pulley and the rod are arranged in a circumferential direction such that rotation positions about the center point of the straight line B and the straight line C are different. Note that the center of the rod may be an area center of the cross section of the rod. When the first condition is satisfied and rotation angles about the center point of the straight line B and the straight line C are sufficiently separated from each other, it is possible to bring the rod closer to the center of the circle, and to realize the reduction in diameter of the surgical tool unit 101. In the present embodiment, the rod and the pulley are alternately arranged in the circumferential direction about the longitudinal axis. Note that the rod and the pulley are not necessarily arranged in the order with regularity in the circumferential direction, and may be arranged in the order of the rod→the pulley→the pulley→the rod→ . . . , and the like.

Furthermore, as a second condition for reducing the diameter of the surgical tool unit 101, the pulley is arranged inside a “straight line A” that is orthogonal at the outermost point to the straight line B connecting the center point and the outermost point. The term “inside” as used herein refers to a side on which the center point of the surgical tool unit 101 exists, of two regions divided by the straight line A, and the other region is “outside” the straight line A. By satisfying the second condition, it is possible to arrange the cable and the pulley near the center point together with the rod in a compact manner. Note that, if the first condition and the second condition are not satisfied and the pulley is arranged on the straight line B outside the straight line A, the layout of the cable and the rod becomes easy, but the diameter of the surgical tool unit 101 increases.

Furthermore, as a third condition for reducing the diameter of the surgical tool unit 101, the rod is arranged such that the straight line C connecting the center point and the center of the rod is shorter than the straight line B connecting the center point of the surgical tool unit 101 and the outermost point of the cable. By further satisfying the third condition while satisfying the first condition or the second condition, the rod is arranged closer to the center point, and the diameter of the surgical tool unit 101 can be further reduced.

### F. Attaching/Detaching Structure, Attaching Procedure, and Detaching Procedure of Surgical Tool Unit

By reducing the size and diameter of the surgical tool unit 101 as described in the above item E, the arm (and the drive unit) at the connection destination and the surgical tool stand can be reduced in size and easily arranged near the robot. Furthermore, when the robot automatically replaces the surgical tool unit, the robot can perform accurate attachment even if the robot roughly inserts the root of the surgical tool unit into the tip end of the drive unit. In this item F, an attaching/detaching structure of the surgical tool unit 101, and an attaching procedure and a detaching procedure to the drive unit 103 will be described.

### F-1. Attaching/Detaching Structure of Surgical Tool Unit

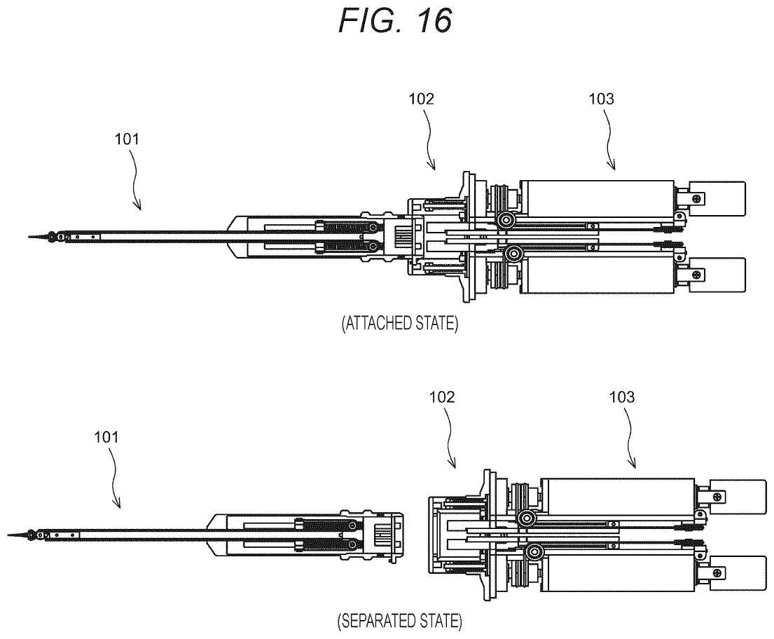

FIG. 16 illustrates cross-sectional views of the respective surgical tool devices 100 in a state where the surgical tool unit 101 is attached to the drive unit 103 and a state where the surgical tool unit 101 is separated from the drive unit 103. In the above item B, as described with reference to the perspective views illustrated in FIGS. 1 and 2, the surgical tool unit 101 is exchangeably attached to the drive unit 103 via the adapter unit 102.

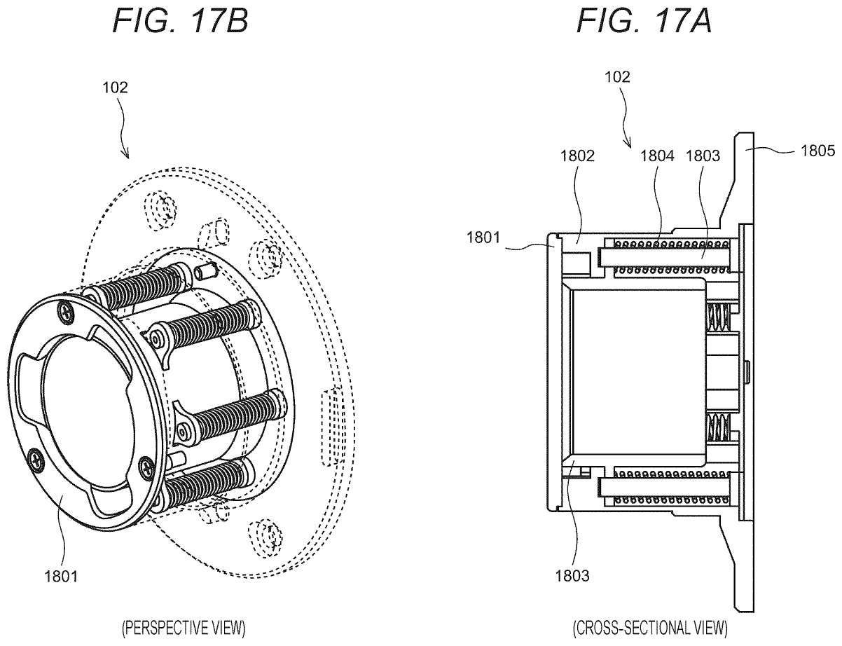

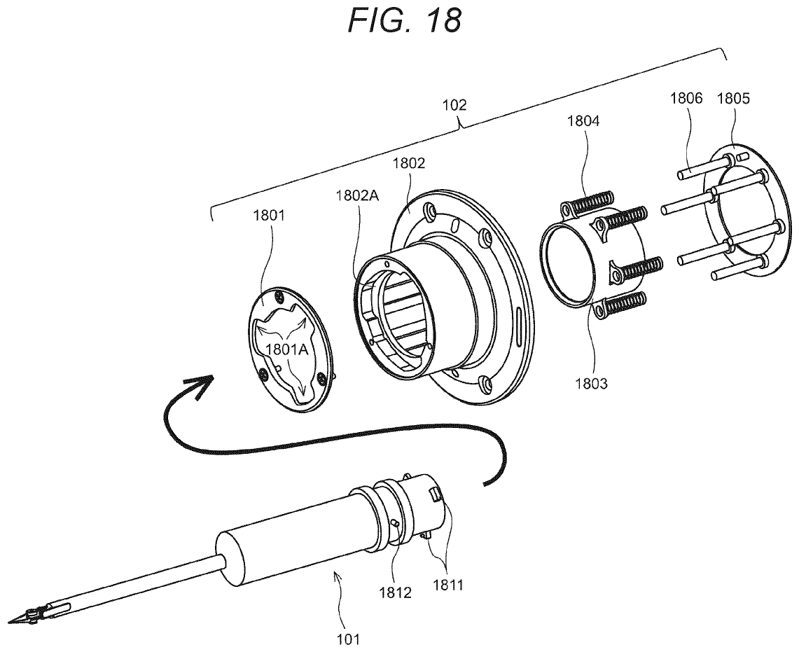

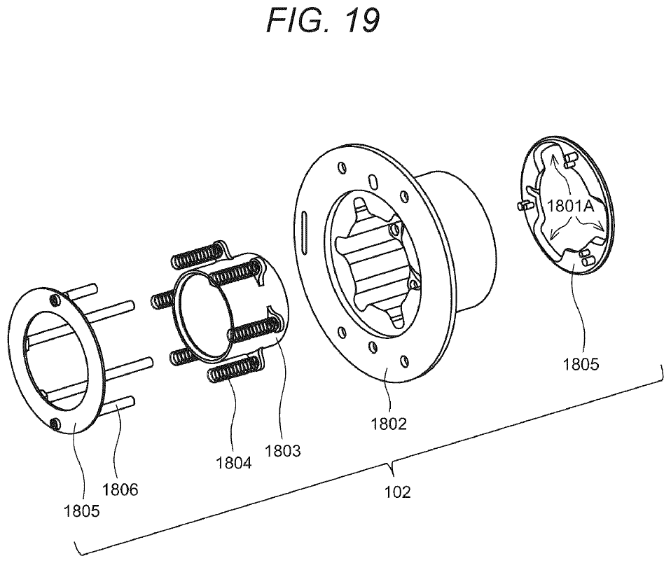

FIGS. 17A and 17B illustrate a cross-sectional view of the adapter unit 102 cut along a plane including the longitudinal axis and a perspective view of the adapter unit 102 as viewed from the distal side (note that, in FIG. 17B, to clarify the internal configuration, a part is made transparent, and the outline is drawn by the dotted line). Furthermore, FIG. 18 is an exploded view of the adapter unit 102 exploded in the longitudinal direction (note that an image to attach the surgical tool unit 101 is also illustrated). For reference, FIG. 19 illustrates an exploded view of the adapter unit 102 as viewed from the proximal side contrary to FIG. 18.

As illustrated in FIG. 18, the adapter unit 102 includes, in order from the distal side, a front plate 1801, an adapter base 1802, a pressing device 1803, a plurality of springs 1804 that pushes out the pressing device 1803 to the distal side, and a spring fixing plate 1805 serving as a contact surface of the other end on the proximal side of each spring 1804. The spring fixing plate 1805 includes a plurality of guide shafts 1806 through which the springs 1804 are inserted to determine positions in an extending and contracting direction.

Furthermore, as illustrated in FIG. 18, the surgical tool unit 101 includes a plurality of claws 1811 and a rotation positioning pin 1812 on an outer periphery on the root side. The front plate 1801 includes a plurality of recesses 1801A for allowing the respective claws 1811 on the surgical tool unit 101 side to pass therethrough, and a positioning groove 1801B that is engaged with the rotation positioning pin 1812 on the surgical tool unit 101 side to determine attachment positions of the surgical tool unit 101 and the adapter unit 102. The pressing device 1803 is biased in the distal direction by the plurality of springs 1804 arranged in the circumferential direction. When each claw 1811 passes through the recess 1801A and the root portion of the surgical tool unit 101 is inserted into the adapter base 1802, the pressing device 1803 presses each claw 1811 of the surgical tool unit 101 against a back surface of the front plate 1801. The adapter base 1802 has a counterbore surface 1802A that comes into contact with each claw 1811 on the surgical tool unit 101 side.

FIG. 20 is an enlarged view of the vicinity of the root of the surgical tool unit 101 attached to the adapter unit 102. Furthermore, the upper part, the interruption, and the lower part of FIG. 21 respectively illustrate front views of states before, in the middle of, and at an attachment position of the surgical tool unit 101 to the adapter unit 102 as viewed from the distal end side.

As illustrated in the upper part of FIG. 21, when alignment is performed such that the longitudinal axes of the surgical tool unit 101 and the adapter unit 102 coincide with each other and each claw 1811 at the root of the surgical tool unit 101 is aligned with the recess 1801A of the front plate 1801 at rotation positions, the surgical tool unit 101 can be inserted into a central opening of the front plate 1801.

Next, as illustrated in the middle part of FIG. 21, when the surgical tool unit 101 is rotated clockwise with respect to the adapter unit 102 toward the attachment position, a clearance between an outer diameter of the claw 1811 and the counterbore surface 1802A of the adapter base 1802 decreases, and the surgical tool unit 101 and the adapter unit 102 are coaxially fixed as illustrated in the lower part of FIG. 21.

### F-2. Attaching Procedure of Surgical Tool Unit

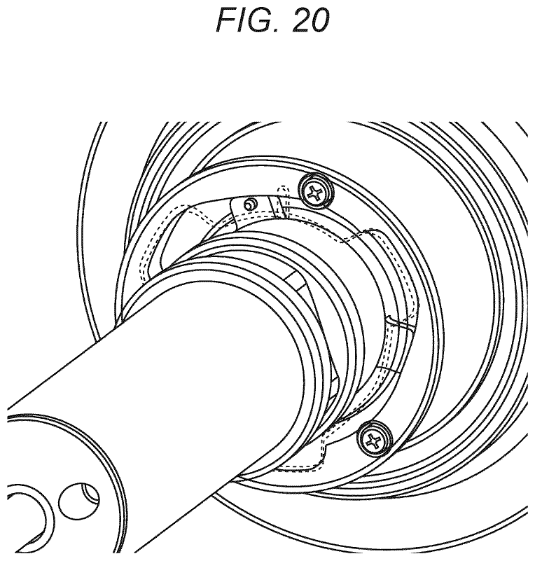

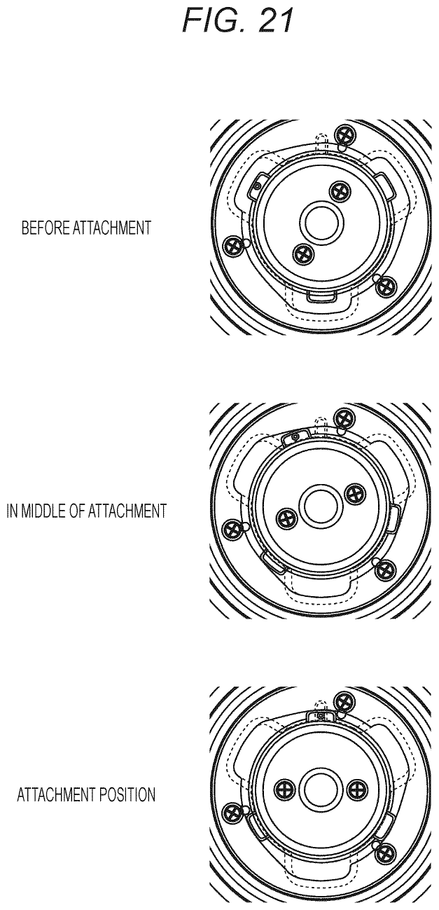

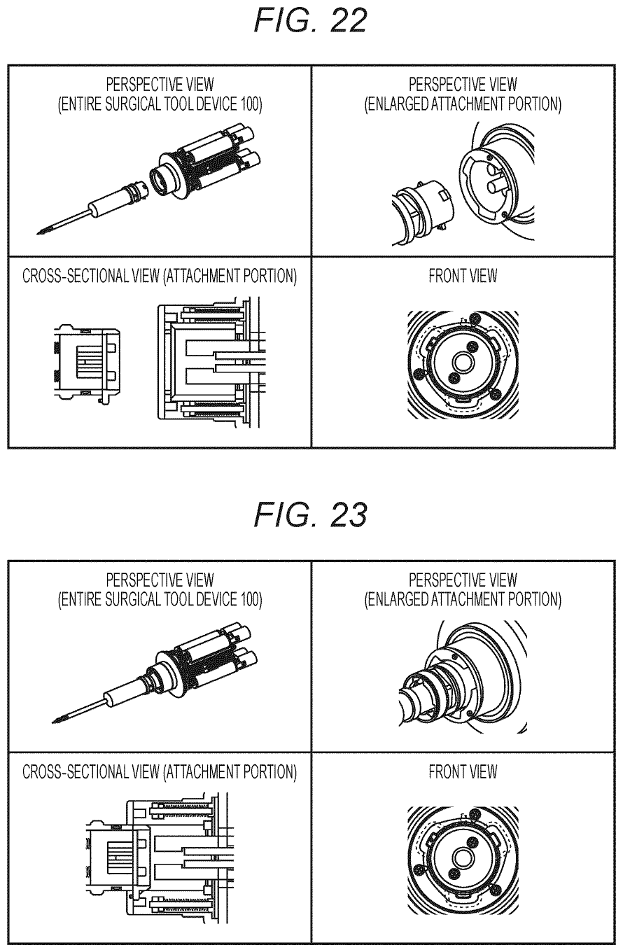

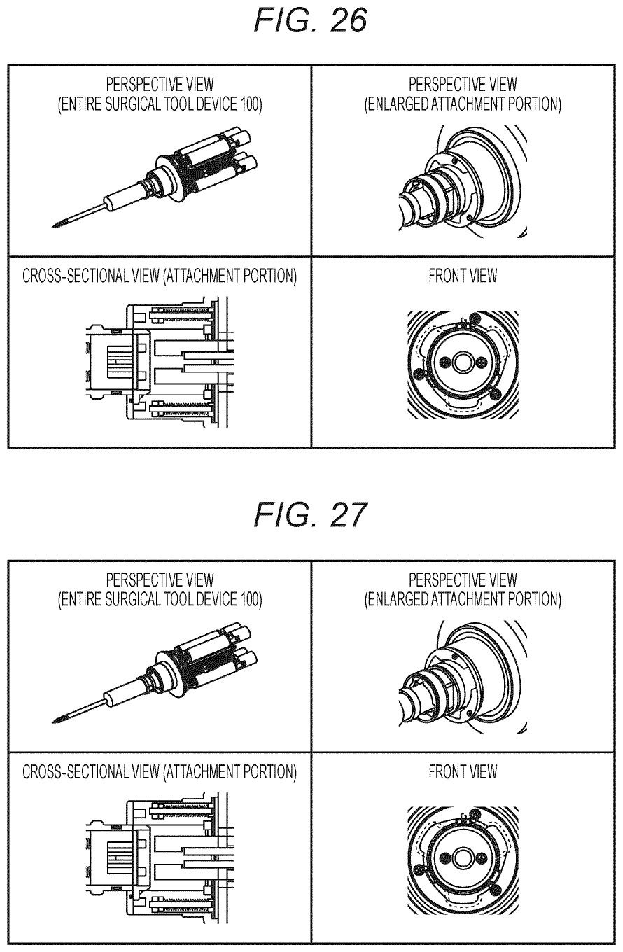

FIGS. 22 to 26 illustrate a perspective view of the entire surgical tool device, a perspective view (enlarged) of an attachment portion, a cross-sectional view of the attachment portion, and a front view as viewed from the distal side when the surgical tool unit 101 is attached to the adapter unit 102. Note that FIG. 22 illustrates a state before attachment, FIGS. 23 to 25 illustrates states in the middle of attachment, and FIG. 26 illustrates a state at the time of completion of attachment.

First, as illustrated in FIG. 22, the claw 1811 on the surgical tool unit 101 side is aligned with the recess 1801A on the adapter unit 102 side (front base 1801) so as to match the rotation positions.

Then, as illustrated in FIG. 23, the claw 1811 on the surgical tool unit 101 side is caused to pass through the recess 1801A on the adapter unit 102 side (front base 1801), and the end portion on the root side of the surgical tool unit 101 is brought into contact with the pressing device 1803.

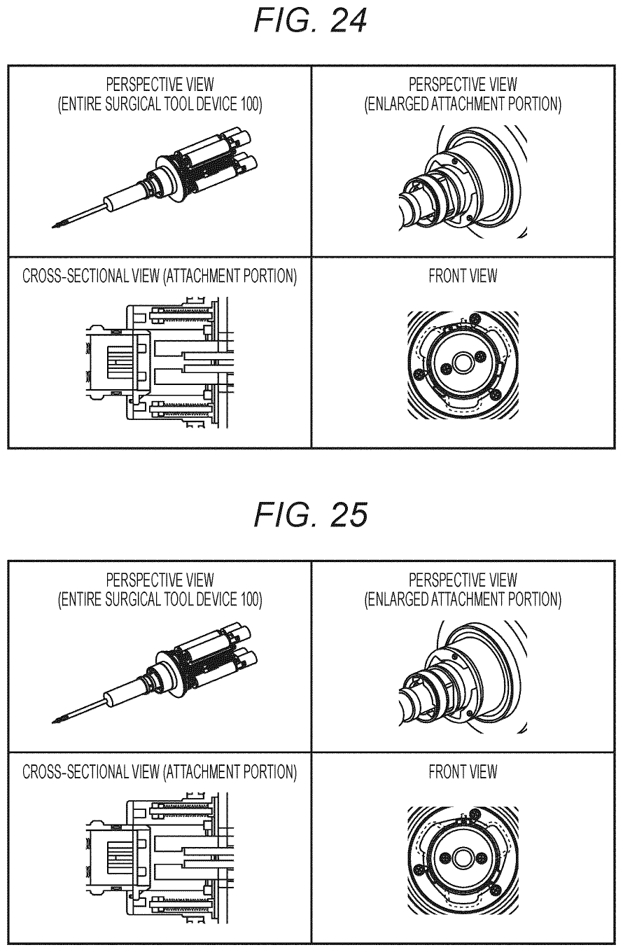

Next, as illustrated in FIGS. 24 and 25, the surgical tool unit 101 is rotated clockwise with respect to the adapter unit 102 toward the attachment position. As a result, the clearance between the outer diameter of the claw 1811 and the counterbore surface 1802A of the adapter base 1802 decreases. Finally, the rotation positioning pin 1812 on the surgical tool unit 101 side is fastened to the positioning groove 1801B of the front plate 1801, the attachment positions of the surgical tool unit 101 and the adapter unit 102 are determined, and the attachment of the surgical tool unit 101 to the adapter unit 102 is completed as illustrated in FIG. 26.

### F-3. Detaching Procedure of Surgical Tool Unit

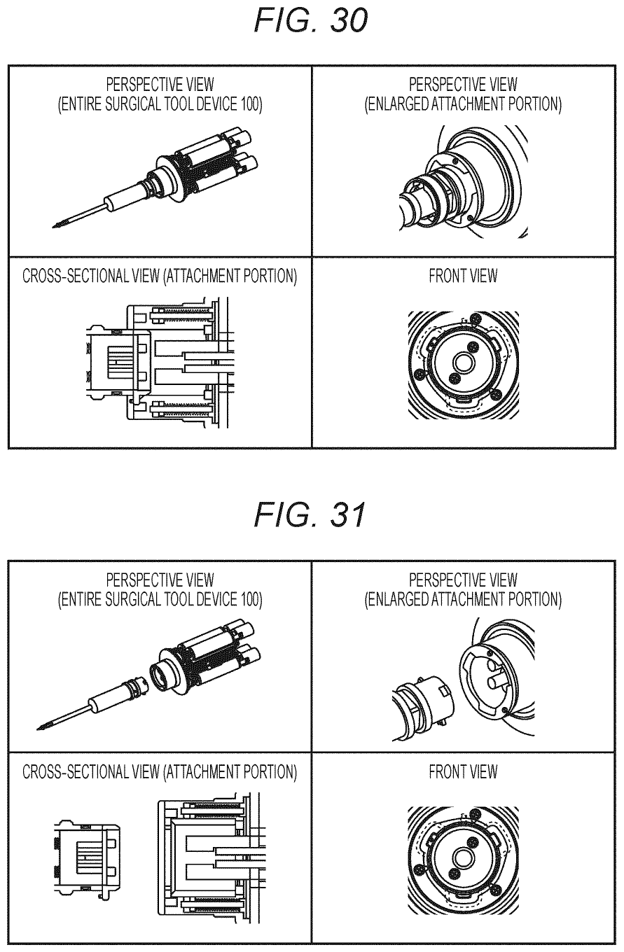

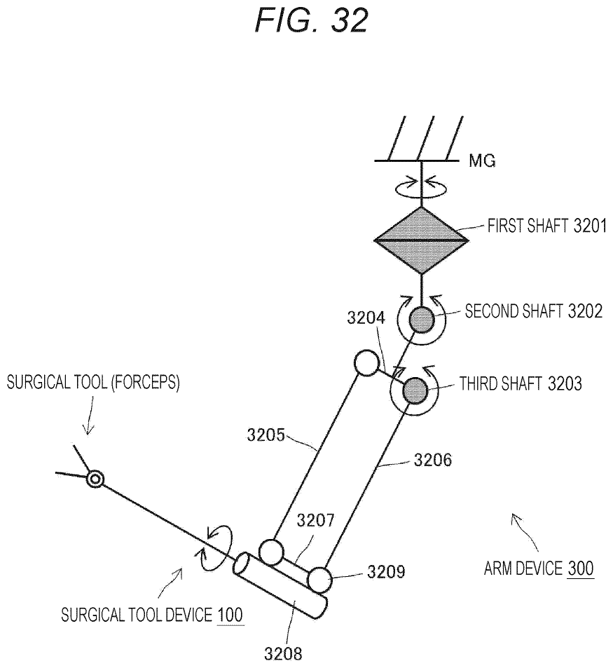

FIGS. 27 to 31 illustrate a perspective view of the entire surgical tool device, a perspective view (enlarged) of an attachment portion, a cross-sectional view of the attachment portion, and a front view as viewed from the distal side when the surgical tool unit 101 is detached from the adapter unit 102. Note that FIG. 27 illustrates a state before detachment, FIGS. 28 to 30 illustrate states in the middle of detachment, and FIG. 31 illustrates a state at the time of completion of detachment.

The state before detachment illustrated in FIG. 27 is the same as the state at the time of completion of attachment illustrated in FIG. 26. First, as illustrated in FIG. 28, the surgical tool unit 101 is pushed to the proximal side until the claw 1811 of the surgical tool unit 101 comes into contact with the counterbore surface 1802A of the adapter base 1802.

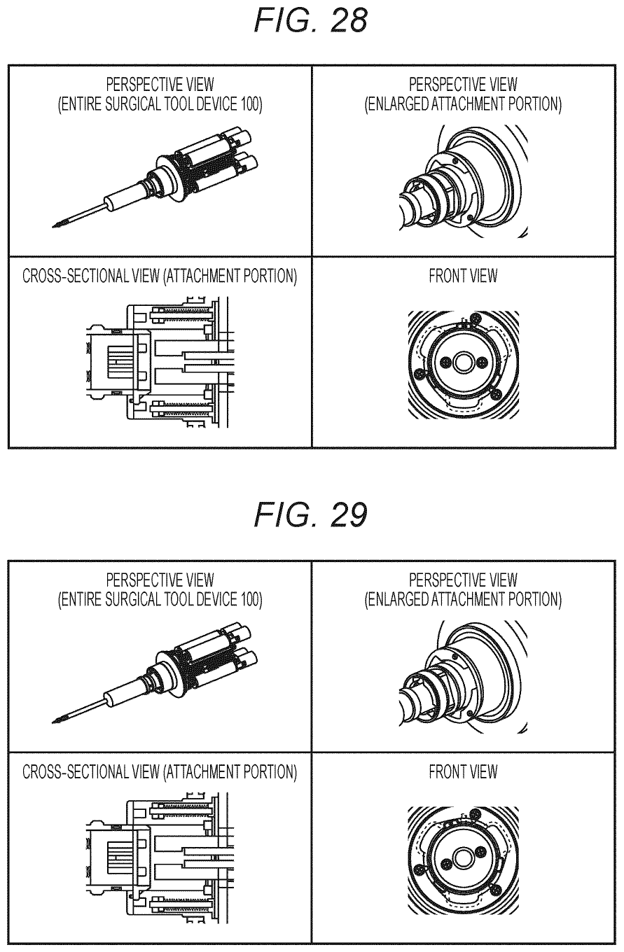

Next, as illustrated in FIG. 29, the surgical tool unit 101 is rotated counterclockwise with respect to the adapter unit 102, and the claw 1811 on the surgical tool unit 101 side is aligned with the recess 1801A on the adapter unit 102 side (front base 1801) to as to match the rotation positions, as illustrated in FIG. 30.

Then, when the claw 1811 on the surgical tool unit 101 side comes to the position where the rotation position is matched with the rotation position of the recess 1801A on the adapter unit 102 side (front base 1801), the surgical tool unit 101 is removed from the adapter unit 102 and the detachment operation is completed as illustrated in FIG. 31.

### G. Arm Device

In the above item B, a point in which the surgical tool device 100 according to the present disclosure is mounted on the arm device 300, and the surgical tool unit 101 is caused to perform the pan operation and the tilt operation has been described with reference to FIG. 3. In this item G, a specific configuration of the arm device 300 and operations of the surgical tool unit 101 mounted on the arm device 300 will be described.

FIG. 32 illustrates a configuration example of the degree of freedom of the arm device 300 illustrated in FIG. 3. For convenience of description, it is assumed that the arm device 300 is suspended from a ceiling that is a mechanical ground (MG). In FIG. 32, only active joints among joint shafts included in the arm device 300 are colored in gray.

The arm device 300 includes, in order from the top, a first shaft 3201 that rotates about a vertical pan axis, a second shaft 3202 that rotates about a horizontal tilt axis, and a four-joint link mechanism including four links 3204 to 3207. The first shaft 3201 and the second shaft 3202 are active shafts.

Among the joints included in the four-joint link mechanism, a third shaft 3203 is an active shaft, and the other joints are passive shafts. Therefore, the four-joint link mechanism includes a prime mover link 3204 driven by the third shaft 3203, two intermediate links 3205 and 3206, and a driven link 3207 that operates following the prime mover link 3204 via the intermediate links 3205 and 3206. Furthermore, the surgical tool device 100 is supported by a device holder 3208 integrated with the driven link 3207. As described above, the surgical tool device 100 includes the surgical tool unit 101, the adapter unit 102, and the drive unit 103, but detailed illustration is omitted in FIG. 32. The device holder 3208 includes a mechanism that rotates the surgical tool device 100 about the longitudinal axis of the shaft 102, but details are omitted.

The first shaft 3201 realizes a pan operation of rotating the entire arm device 300 about the vertical pan axis with respect to the mechanical ground. Furthermore, the second shaft 3203 couples an output shaft of the first shaft and the four-joint link mechanism, and realizes a first tilt operation of rotating the entire four-joint link mechanism about the tilt axis. Furthermore, the third shaft 3203 can rotate the prime mover link 3204 about the third shaft 3203 to cause the driven link 3207 to follow the rotation in the four-joint link mechanism. As a result, the third shaft 3203 realizes a second tilt operation of rotating the surgical tool device 100 supported by the device holder 3208 integrated with the driven link 3207 about the joint shaft 3209 at the lowermost end.

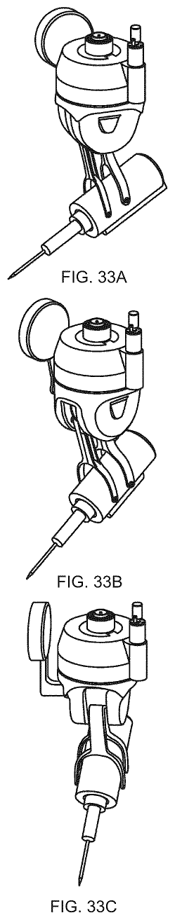

FIGS. 33(A) to 33(C) illustrate a series of operations in which the arm device 300 pans the surgical tool device 100. The arm device 300 can cause the surgical tool device 100 to perform the pan operation about the first shaft 3201 by driving the first shaft 3201.

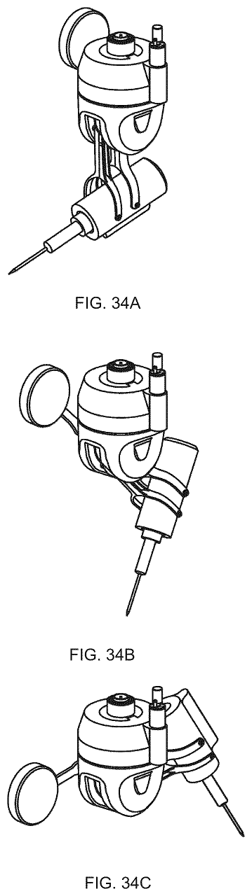

Furthermore, FIGS. 34(A) to 34(C) illustrate a series of operations in which the arm device 300 tilts the surgical tool device 100 with respect to the main body of the arm device 300. When the second shaft 3203 rotates, the entire four-joint link mechanism is rotated about the tilt axis, and the surgical tool device 100 can perform the tilt operation about the second shaft 3203 (this is referred to as “first tilt operation”).

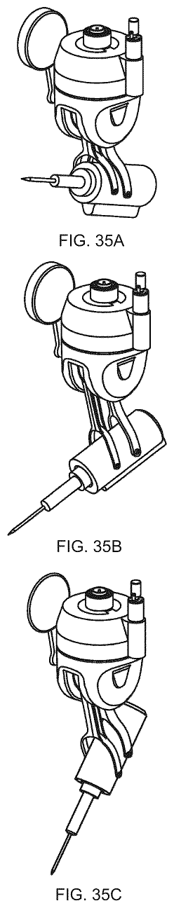

Furthermore, FIGS. 35(A) to 35(C) illustrate a series of operations in which the arm device 300 tilts the surgical tool device 100 at the current position. When the third shaft 3203 rotates, the prime mover link 3204 rotates about the third shaft 3203, and the driven link 3207 rotates about the joint shaft 3209 so as to follow the prime mover link 3204. As a result, the surgical tool device 100 supported by the device holder 3208 integrated with the driven link 3207 can be rotated about the joint shaft 3209 at the lowermost end (this is referred to as a “second tilt operation”).

### H. Master-Slave System

In general, a surgical operation is a difficult task performed by sensory movement of an operator. Recently, a master-slave surgical system has been introduced in order to suppress tremor of the operator and realize precise surgery. The arm device 300 described in the above item G can be applied to a master-slave system as a slave robot remotely controlled from a master side.

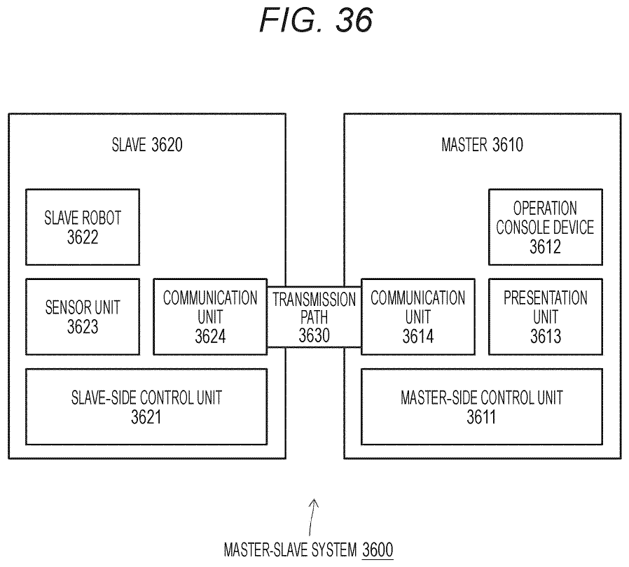

FIG. 36 schematically illustrates a functional configuration example of a master-slave system 3600. The illustrated master-slave system 3600 includes a master 3610 that remotely controls a slave robot and a slave 3620 having the slave robot. In a case where the master-slave system 3600 is applied to surgery, a user such as an operator operates an operation console device on the side of the master 3610, and driving of the slave robot 3622 including the arm device 300 and the like is controlled according to the operation of the user on the side of the slave 3620 installed in an operating room, so that the surgery can be performed.

The master 3610 is installed outside the operating room (alternatively, a place separated from an operating table in the operating room), for example, and the user (operator) remotely operates the slave 3620. The slave 3620 includes a slave robot 3622 such as the arm device 300 installed near the operating table. The arm device 300 supports the surgical tool device 100 as an end effector including a surgical tool and an observation device, and realizes the pan operation, the tilt operation, and the like as described in the above item G. The surgical tool referred to herein is, for example, a medical instrument such as a pair of forceps, a pneumoperitoneum tube, an energy treatment tool, a pair of tweezers, or a retractor, and the observation device is, for example, an endoscope. Then, the slave robot 3622 performs surgery for a patient laid on the operating table in accordance with an instruction from the master 3610. Examples of the surgery described herein include a laparoscopic surgery, a celoscopic surgery, a brain surface surgery, and an eyeball or eyeground surgery. The master 3610 and the slave 3620 are interconnected via a transmission path 3630. The transmission path 3630 is desirably capable of performing signal transmission with a low delay using, for example, a medium such as an optical fiber.

The master 3610 includes a master-side control unit 3611, an operation console device 3612, a presentation unit 3613, and a master-side communication unit 3614. The master 3610 operates under the overall control of the master-side control unit 3611.

The operation console device 3612 is an input device for a user (operator or the like) to perform a remote operation or an on-screen 3D operation for a slave robot 3622 that mounts a surgical tool such as forceps in the slave 3620. It is assumed that the operation console device 3612 can perform operations of three degrees of freedom in translation for translating the surgical tool, three degrees of freedom in rotation for changing a posture of the surgical tool, and one degree of freedom in gripping such as an opening/closing operation of the forceps, for example.

The presentation unit 3613 presents information regarding surgery performed in the slave 3620 to a user (operator) operating the operation console device 3612 on the basis of sensor information mainly acquired by a sensor unit 3623 (to be described below) on the slave 3620 side.

For example, in a case where the sensor unit 3623 on the slave 3620 side is equipped with an RGB camera for observing a surface of an affected part, an RGB camera for capturing a microscopic image, an endoscope in laparoscopic or celoscopic surgery, or an interface for capturing captured images of these cameras, and these image data are transferred to the operation console device 3612 with a low delay through the transmission path 3630, the presentation unit 3613 displays the captured image of the affected part of the affected part in real time on a screen using a monitor display or the like.

Furthermore, in a case where the sensor unit 3623 is equipped with a function to measure a force sense such as an external force or a moment acting on the surgical tool mounted on the slave robot 3622, and such force sense information is transferred to the master 3610 with a low delay via the transmission path 3630, the presentation unit 3613 performs force sense presentation to the user (operator). The force sense presentation function of the presentation unit 3613 is incorporated and implemented in the operation console device 3612. Specifically, the presentation unit 3613 performs the force sense presentation to the user (operator) by driving a grip portion having, for example, three degrees of freedom in rotation and one degree of freedom in gripping of the tip end of the operation console device 3612 with a motor.

The master-side communication unit 3614 performs a signal transmission/reception process with the slave 3620 via the transmission path 3630 under the control of the master-side control unit 3611. For example, in a case where the transmission path 3630 includes an optical fiber, the master-side communication unit 3614 includes an electro-optical conversion unit that converts an electrical signal transmitted from the master 3610 into an optical signal, and a photoelectric conversion unit that converts an optical signal received from the transmission path 3630 into an electrical signal. The master-side communication unit 3614 transfers an operation command for the slave robot 3622 input by the user (operator) via the master 3610 to the slave 3620 via the transmission path 3630. Furthermore, the master-side communication unit 3614 receives the sensor information transmitted from the slave 3620 via the transmission path 3630.

On the other hand, the slave 3620 includes a slave-side control unit 3621, a slave robot 3622, a sensor unit 3623, and a slave-side communication unit 3624. The slave 3620 operates in accordance with an instruction from the master 3610 under the overall control of the slave-side control unit 3621.

The slave robot 3622 is, for example, an arm type surgical robot having an articulated link structure such as the above-described arm device 300, and is mounted with a surgical tool as an end effector and an observation device at the tip end (or the distal end). Examples of the surgical tools include forceps, a pneumoperitoneum tube, an energy treatment tool, tweezers, and a retractor. Furthermore, an example of the observation device includes an endoscope. The slave-side control unit 3621 interprets the operation command transmitted from the master 3610 via the transmission path 3630, converts the operation command into a drive signal of an actuator that drives the slave robot 3622, and outputs the drive signal. The slave robot 3622 then operates on the basis of the drive signal from the slave-side control unit 3621.

The sensor unit 3623 includes a plurality of sensors for detecting a status in an affected part of the operation performed by the slave robot 3622 or the slave robot 3622, and further includes an interface for taking in sensor information from various sensor devices installed in the operating room. For example, the sensor unit 3623 includes a force sense sensor (force torque sensor: FTS) for measuring the external force or the moment acting during the operation on the surgical tool mounted at the tip end (distal end) of the slave robot 3622. Furthermore, the sensor unit 3623 is equipped with an observation device such as an RGB camera for observing a surface of an affected part during surgery by the slave robot 3622, an RGB camera for capturing a microscopic image, or an endoscope in laparoscopic or celoscopic surgery, or is equipped with an interface for capturing captured images of these cameras.

The slave-side communication unit 3624 performs a signal transmission/reception process with the master 3610 via the transmission path 3630 under the control of the slave-side control unit 3621. For example, in a case where the transmission path 3630 includes an optical fiber, the slave-side communication unit 3624 includes an electro-optical conversion unit that converts an electrical signal transmitted from the slave 3620 into an optical signal, and a photoelectric conversion unit that converts an optical signal received from the transmission path 3630 into an electrical signal.

The slave-side communication unit 3624 transfers force sense data of the surgical tool acquired by the sensor unit 3623, and captured images of an RGB camera for observing the surface of the affected part, an RGB camera for capturing a microscopic image, an endoscope like in laparoscopic or celoscopic surgery, and the like to the operation console device 3612 through the transmission path 3630. Furthermore, the slave-side communication unit 3624 receives the operation command on the slave robot 3622 transmitted from the master 3610 via the transmission path 3630.

On the master 3610 side, an operation command for remotely operating the slave robot 3622 is input via the operation console device 3612. The operation command includes the pan operation of the arm device 300 (see FIG. 33), the first tilt operation of the arm device 300 (see FIG. 34), the second tilt operation of the arm device 300 (see FIG. 35), a rotation operation about the longitudinal axis (or roll axis) of the surgical tool device 100 held by the arm device 300, and an operation of the surgical tool at the distal end of the surgical tool unit 101.

The slave-side control unit 3621 performs drive control of the active shafts (the first shaft to the third shaft) of the arm device 300 and drive control of the drive device 103 so as to realize the operations of the arm device 300 and the surgical tool unit 101 according to the received operation command.

On the master 3610 side, the operation of the arm device 300 (see FIGS. 19 to 21) and the operation of the surgical tool (specifically, a yaw operation, a pitch operation, and an opening/closing operation of the forceps) are instructed via the operation console device 3612. In a case of receiving the operation command instructing the yaw operation, the pitch operation, or the opening/closing operation of the forceps from the master 3610 side, the slave-side control unit 3621 calculates a rotation angle of each motor in the drive unit 103 for realizing the instructed operation of the forceps, and generates an angle command to each motor.

A fixed input/output relationship is established between the rotation angle of the motor on the drive unit 103 side and each operation such as opening/closing, pitch rotation, or yaw rotation of the forceps of the surgical tool unit 101. Since the surgical tool unit 101 to which the present disclosure is applied is backlashless and has high back drivability, the forceps at the distal end of the surgical tool unit 101 can be accurately driven on the basis of the rotation angle of the motor on the drive unit 103 side.

Furthermore, in a case where the forceps receives an external force from the outside (affected part or the like) and the jaws are displaced during the operation of the slave robot 3622, a displacement amount of the jaws is converted from a displacement angle of each motor on the basis of a detection result of an encoder of each motor, and is sent to the master 3610 side as force sense feedback information. Since the drive unit 103 to which the present embodiment is applied has high back drivability, the displacement amount of the surgical tool can be measured with high accuracy on the basis of the displacement angle of the motor. Therefore, accurate force sense feedback information can be supplied to the master 3610 side to realize precise surgery.

### INDUSTRIAL APPLICABILITY

The present disclosure has been described in detail with reference to a specific embodiment. However, it is obvious that those skilled in the art can make modifications and substitutions of the embodiment without departing from the scope of the present disclosure.

In the present specification, the embodiment in which the present disclosure is mainly applied to surgery in the medical field has been mainly described, but the gist of the present disclosure is not limited thereto. The present disclosure can be applied to a wide variety of fields such as a remote operation robot that performs precise work in a difficult-to-work space such as a manufacturing factory, a construction site, or outer space, and an operation console device for remote operation, and can reduce the diameter and size of the end effector unit. As a result, it becomes easy to install a plurality of types of end effector units on a mounting table (surgical tool stand or the like) in the vicinity of the robot, and the robot itself can automatically replace the end effector unit in a short time.

In short, the present disclosure has been described in an illustrative manner, and the contents disclosed in the present specification should not be interpreted in a limited manner. To determine the subject matter of the present disclosure, the claims should be taken into consideration.

Note that the present disclosure may also have the following configurations.

(1) An end effector unit including:
a cable having one end on a tip end side connected to an end effector;
a pulley configured to fold back the cable extending toward a root side in a tip end direction; and
a linear motion transmission device having one degree of freedom in linear traveling in a longitudinal direction and connected to the other end of the cable folded back by the pulley.

(1-1) The end effector unit according to (1) described above, further including:
a shaft that supports the end effector at a distal end and through which the cable is inserted.

(2) The end effector unit according to (1) described above, in which
the pulley is arranged such that a first straight line connecting a longitudinal axis of the end effector unit and an outermost point of the cable folded back by the pulley, the outermost point being farthest from the longitudinal axis, and a second straight line connecting the longitudinal axis and a center of the linear motion transmission device have different rotation positions about the longitudinal axis.

(3) The end effector unit according to (1) described above, in which
the pulley is arranged on a side of a longitudinal axis of the end effector unit with respect to a third straight line orthogonal at an outermost point to a first straight line connecting the longitudinal axis of the end effector unit and the outermost point of the cable folded back by the pulley, the outermost point being farthest from the longitudinal axis.

(4) The end effector unit according to (1) described above, in which
the pulley is arranged such that a second line segment connecting a longitudinal axis of the end effector unit and a center of the linear motion transmission device is shorter than a first line segment connecting the longitudinal axis and an outermost point of the cable folded back by the pulley, the outermost point being farthest from the longitudinal axis.

(5) The end effector unit according to any one of (1) to (4) described above, in which
the linear motion transmission device includes a rod having one degree of freedom in linear traveling in the longitudinal direction.

(6) The end effector unit according to any one of (1) to (5) described above, in which
the linear motion transmission device and the pulley are alternately arranged in a circumferential direction around a longitudinal axis.

(7) The end effector unit according to (5) or (6) described above, in which
the linear motion transmission device further includes a rotation suppression device that suppresses rotation of the rod about a longitudinal axis.

(8) The end effector unit according to any one of (1) to (6) described above, further including:
a base configured to support the linear motion transmission device so as to slide in the longitudinal direction; and
a rotation suppression device attached to the linear motion transmission device and configured to suppress rotation of the linear motion transmission device about a longitudinal axis.

(9) The end effector unit according to (8) described above, in which
a guide groove formed along the longitudinal direction in one of the base and the rotation suppression device, and a protrusion to be inserted into the guide groove and formed in the other of the base and the rotation suppression device are included, and
the guide groove guides the protrusion to suppress the rotation of the linear motion transmission device about the longitudinal axis.

(10) The end effector unit according to (8) or (9) described above, in which
the rotation suppression device includes a cable coupling unit that connects the other end of the cable.

(11) The end effector unit according to any one of (8) to (10) described above, in which
the linear motion transmission device is guided in linear traveling motion by at least two bearings arranged in the longitudinal direction, and
the rotation suppression device is attached to the linear motion transmission device at an intermediate position between positions where the rotation suppression device is supported by any two of the bearings.

(12) The end effector unit according to (11) described above, in which
the bearing includes a plain bearing.

(13) The end effector unit according to any one of (1) to (12) described above, further including:
a tension applying portion that applies an external force to the linear motion transmission device in a direction in which a tension is applied to the cable.

(14) The end effector unit according to (13) described above, in which
the tension applying portion includes a spring arranged between a base that supports the linear motion transmission device and a rotation suppression device attached to a predetermined position in the longitudinal direction of the linear motion transmission device.

(15) The end effector unit according to any one of (1) to (14) described above, in which
the end effector has two bending axes and a degree of freedom in gripping, and operates by a driving force transmitted by four cables.

(16) The end effector unit according to (15) described above, in which
the four cables are respectively folded back in the tip end direction by corresponding pulleys and then respectively connected to corresponding linear motion transmission devices.

(17) The end effector unit according to any one of (1) to (16) described above, in which
the end effector includes a surgical tool used for surgery.

(18) A surgical tool device including:
a surgical tool unit including a surgical tool supported at a distal end, a cable having one end on a tip end side connected to the surgical tool, a pulley that folds back the cable extending to a root side in a tip end direction, and a linear motion transmission device having one degree of freedom in linear traveling in a longitudinal direction and connected to the other end of the cable folded back by the pulley; and
a drive unit to which the surgical tool unit is attached and configured to drive the linear motion transmission device.

(19) An arm device including:
a surgical tool device including a surgical tool unit including a surgical tool supported at a distal end, a cable having one end on a tip end side connected to the surgical tool, a pulley that folds back the cable extending to a root side in a tip end direction, and a linear motion transmission device having one degree of freedom in linear traveling in a longitudinal direction and connected to the other end of the cable folded back by the pulley, and a drive unit to which the surgical tool unit is attached and configured to drive the linear motion transmission device; and
an arm of an articulated link structure that supports the surgical tool device.

(20) A master-slave system including:
a slave device including a surgical tool device including a surgical tool unit including a surgical tool supported at a distal end, a cable having one end on a tip end side connected to the surgical tool, a pulley that folds back the cable extending to a root side in a tip end direction, and a linear motion transmission device having one degree of freedom in linear traveling in a longitudinal direction and connected to the other end of the cable folded back by the pulley, and a drive unit to which the surgical tool unit is attached and configured to drive the linear motion transmission device, and an arm of an articulated link structure that supports the surgical tool device; and
a master device configured to operate the surgical tool device and the arm.

### REFERENCE SIGNS LIST

101 Surgical tool unit
102 Adapter unit
103 Drive unit
300 Arm device
401 Surgical tool
402 Shaft
403 Surgical tool unit base
411, 421 Cable
412, 422 Pulley
413, 423 Rod
414, 424 Spring
501 Drive unit base
511, 521 Motor
512, 522 Speed reducer
513, 523 Encoder
514, 524 Motor capstan
515, 516, 525, 526 Cable
515A, 516A, 525A, 526A Idler pulley
517, 527 Rod
518, 528 Linear guide
701 Surgical tool
702 Shaft
703 Inner base
704 Joint unit
705 Case
711, 721, 731, 741 Cable
712, 722, 732, 742 Pulley
713, 723, 733, 743 Rod
714 Rotation suppression device
714A Protrusion
715 Spring
811, 812, 821, 822 Bearing (plain bearing)
910 Shaft connection portion
920 Rod support portion
921 to 924 Rod insertion hole
930 Frame
931 Guide groove
940 Pulley support portion
1801 Front plate
1801A Recess
1801B Positioning groove
1802 Adapter base
1802A Counterbore surface
1803 Pressing device
1804 Spring
1805 Spring fixing plate
1806 Guide shaft
1811 Claw
1812 Positioning pin
3600 Master-slave system
3610 Master
3611 Master-side control unit
3612 Operation console device
3613 Presentation unit
3614 Master-side communication unit
3620 Slave
3621 Slave-side control unit
3622 Slave robot
3623 Sensor unit
3624 Slave-side communication unit
3630 Transmission path

1. An end effector unit comprising:
a cable having one end on a tip end side connected to an end effector;
a pulley configured to fold back the cable extending toward a root side in a tip end direction; and
a linear motion transmission device having one degree of freedom in linear traveling in a longitudinal direction and connected to the other end of the cable folded back by the pulley.

2. The end effector unit according to claim 1, wherein
the pulley is arranged such that a first straight line connecting a longitudinal axis of the end effector unit and an outermost point of the cable folded back by the pulley, the outermost point being farthest from the longitudinal axis, and a second straight line connecting the longitudinal axis and a center of the linear motion transmission device have different rotation positions about the longitudinal axis.

3. The end effector unit according to claim 1, wherein
the pulley is arranged on a side of a longitudinal axis of the end effector unit with respect to a third straight line orthogonal at an outermost point to a first straight line connecting the longitudinal axis of the end effector unit and the outermost point of the cable folded back by the pulley, the outermost point being farthest from the longitudinal axis.

4. The end effector unit according to claim 1, wherein
the pulley is arranged such that a second line segment connecting a longitudinal axis of the end effector unit and a center of the linear motion transmission device is shorter than a first line segment connecting the longitudinal axis and an outermost point of the cable folded back by the pulley, the outermost point being farthest from the longitudinal axis.

5. The end effector unit according to claim 1, wherein
the linear motion transmission device includes a rod having one degree of freedom in linear traveling in the longitudinal direction.

6. The end effector unit according to claim 1, wherein
the linear motion transmission device and the pulley are alternately arranged in a circumferential direction around a longitudinal axis.

7. The end effector unit according to claim 5, wherein
the linear motion transmission device further includes a rotation suppression device that suppresses rotation of the rod about a longitudinal axis.

8. The end effector unit according to claim 1, further comprising:
a base configured to support the linear motion transmission device so as to slide in the longitudinal direction; and
a rotation suppression device attached to the linear motion transmission device and configured to suppress rotation of the linear motion transmission device about a longitudinal axis.

9. The end effector unit according to claim 8, wherein
a guide groove formed along the longitudinal direction in one of the base and the rotation suppression device, and a protrusion to be inserted into the guide groove and formed in the other of the base and the rotation suppression device are included, and
the guide groove guides the protrusion to suppress the rotation of the linear motion transmission device about the longitudinal axis.

10. The end effector unit according to claim 8, wherein
the rotation suppression device includes a cable coupling unit that connects the other end of the cable.

11. The end effector unit according to claim 8, wherein
the linear motion transmission device is guided in linear traveling motion by at least two bearings arranged in the longitudinal direction, and
the rotation suppression device is attached to the linear motion transmission device at an intermediate position between positions where the rotation suppression device is supported by any two of the bearings.

12. The end effector unit according to claim 11, wherein
the bearing includes a plain bearing.

13. The end effector unit according to claim 1, further comprising:
a tension applying portion that applies an external force to the linear motion transmission device in a direction in which a tension is applied to the cable.

14. The end effector unit according to claim 13, wherein
the tension applying portion includes a spring arranged between a base that supports the linear motion transmission device and a rotation suppression device attached to a predetermined position in the longitudinal direction of the linear motion transmission device.

15. The end effector unit according to claim 1, wherein
the end effector has two bending axes and a degree of freedom in gripping, and operates by a driving force transmitted by four cables.

16. The end effector unit according to claim 15, wherein
the four cables are respectively folded back in the tip end direction by corresponding pulleys and then respectively connected to corresponding linear motion transmission devices.

17. The end effector unit according to claim 1, wherein
the end effector includes a surgical tool used for surgery.

18. A surgical tool device comprising:
a surgical tool unit including a surgical tool supported at a distal end, a cable having one end on a tip end side connected to the surgical tool, a pulley that folds back the cable extending to a root side in a tip end direction, and a linear motion transmission device having one degree of freedom in linear traveling in a longitudinal direction and connected to the other end of the cable folded back by the pulley; and
a drive unit to which the surgical tool unit is attached and configured to drive the linear motion transmission device.

19. An arm device comprising:
a surgical tool device including a surgical tool unit including a surgical tool supported at a distal end, a cable having one end on a tip end side connected to the surgical tool, a pulley that folds back the cable extending to a root side in a tip end direction, and a linear motion transmission device having one degree of freedom in linear traveling in a longitudinal direction and connected to the other end of the cable folded back by the pulley, and a drive unit to which the surgical tool unit is attached and configured to drive the linear motion transmission device; and
an arm of an articulated link structure that supports the surgical tool device.

20. A master-slave system comprising:
a slave device including a surgical tool device including a surgical tool unit including a surgical tool supported at a distal end, a cable having one end on a tip end side connected to the surgical tool, a pulley that folds back the cable extending to a root side in a tip end direction, and a linear motion transmission device having one degree of freedom in linear traveling in a longitudinal direction and connected to the other end of the cable folded back by the pulley, and a drive unit to which the surgical tool unit is attached and configured to drive the linear motion transmission device, and an arm of an articulated link structure that supports the surgical tool device; and
a master device configured to operate the surgical tool device and the arm.
